(19) United States
(12) Patent Application Publication
(10) Pub. No.: US 2007/0073271 A1
(43) Pub. Date: Mar. 29, 2007

(54) Catheter for diagnostic imaging and therapeutic procedures

(76) Inventors: Gregory G. Brucker, Minneapolis, MN (US); Steven D. Savage, Paynesville, MN (US); John R. Gardner, Wexford, PA (US); Frederick W. Trombley, III, Gibsonia, PA (US)

Correspondence Address: GREGORY L BRADLEY;MEDRAD INC, ONE MEDRAD DRIVE, INDIANOLA, PA, 15051, US

(21) Appl. No.: 10/575,318

(22) PCT Filed: Nov. 15, 2004
(86) PCT No.: PCT/US04/38093

§ 371 Date: Apr. 11, 2006

Related U.S. Application Data
(60) Provisional application No. 60/520,071, filed on Nov. 15, 2003.

(51) Int. Cl. A61M 25/16; A61M 25/18

(52) U.S. Cl. 604/537

(57) ABSTRACT

A catheter for use in introducing fluid into a vessel or other bodily structure. The catheter comprises a stem and a restrictor. The stem has approximate a distal end thereof a porous section that defines microholes distributed thereabout, which are inclined by a predetermined angle in the proximal direction. Affixed to the stem, the restrictor includes a conically-shaped valve with an apex thereof defining an opening and pointing in the proximal direction. The opening generally decreases in size as the conically-shaped valve flattens out distally as the pressure of the fluid within the tip increases. The forces of the fluid flowing out of the opening of the restrictor and out of the microholes of the stem substantially balance thereby substantially eliminating both recoil and whipping of the catheter, thus enabling its position to remain exceptionally stable while the fluid is finely dispersed therefrom in a cloud-like form.

### CROSS-REFERENCE TO RELATED APPLICATION

This application claims the benefit of U.S. Provisional Application No. 60/520,071, filed 15 Nov. 2003, which is incorporated herein by reference.

### FIELD OF THE INVENTION

The invention relates generally to catheters used for diagnostic imaging, therapeutic treatments, drug delivery, perfusion, and various other interventional procedures that require delivery of fluids into the vasculature or other structures of a patient. More particularly, the invention pertains to a catheter having an innovative distal end whose position remains exceptionally stable within the vasculature or other structure while the fluid is very finely dispersed therefrom during such procedures.

### BRIEF DESCRIPTION OF RELATED ART

The following information is provided to assist the reader to understand the invention disclosed below and at least some of the many applications in which it will typically be used. It is also provided to inform the reader of at least some of the many different types, shapes and sizes of catheters to which the invention can be applied. In addition, any references set forth herein are intended merely to assist in such understanding. Inclusion of a reference herein, however, is not intended to and does not constitute an admission that the reference is available as prior art with respect to the invention.

As is well known, a catheter is a flexible, tube-shaped surgical instrument for introducing fluids into, or withdrawing fluids from, vessels and various other structures in the body. Catheters come in many different types, shapes and sizes, and, considered collectively, they are used for many different purposes. They are often loosely named or categorized according to the vessel or other structure to which they are applied or the specific use to which they are put. As an example of the former, “venous catheters” are inserted into veins and are typically used in connection with therapeutic procedures. “Arterial catheters” are inserted into arteries and, as they are often used for diagnostic imaging, they are often referred to as diagnostic catheters (though they are also used for administering therapeutic agents). As an example of the latter, “infusion catheters” are used for infusing an infusate (e.g., a therapeutic agent or a diagnostic agent) into veins, arteries or other structures in the body.

The process of inserting a catheter is referred to as catheterization. Placement of a catheter into a particular vessel or structure may, for example, allow a clinician: (i) to remove fluids from the body {e.g., urine can be drained from the bladder via urinary catheterization}; (ii) to infuse anesthetics and other drugs to anesthetize patients before certain medical procedures; (iii) to directly measure blood pressure in an artery or vein; (iv) to administer therapeutic agents, intravenous fluids, medication or parenteral nutrition; and (v) to inject dye or contrast media into blood vessels or other structures to visualize abnormalities {e.g., in the heart via cardiac catheterization}. The invention disclosed herein is primarily discussed in connection with catheters designed for the latter three applications, though it may also be equally applicable to other applications.

As shown in FIGS. 1A-1E, for example, catheters come in different diameters quantified in “French” (⅓ mm), various lengths quantified in centimeters, and varied geometric shapes often designated by specific names. Diagnostic catheters typically range from 3 to 9 French in diameter and 60 to 130 cm in length. Shapes are typically classified as “coronary” if they are associated with the arteries of the heart, or “peripheral” or “radiology” if associated with the arteries and veins of the peripheral vasculature. For the heart, the shapes include, for example, the Judkins Right (JR), the Amplatz Right (AR) and the Right Coronary Bypass (RCB) for the right coronary artery; the Judkins Left (JL), the Amplatz Left (AL) and the Left Coronary Bypass (LCB) for the left coronary artery; and the Pigtail Straight and Angulated for the ventricles (ventriculogram) and the aorta (aortogram). Examples of these catheters, in various shapes and sizes, are shown in FIGS. 1B-1E. For the peripheral arteries, the shapes include the Visceral, the Cobra, and the RDC shaped catheters for the renal arteries; and the Simmons, the JB, and the Headhunter shapes for the carotid arteries.

FIG. 1A illustrates a prior art catheter of the type used in various cardiac procedures. Used typically for diagnostic imaging applications, this cardiovascular catheter has five basic elements. The hub, located at the proximal end, is the interface with both the clinician and the various medical devices to which it can be attached, typically via a Luer connector. The hub is the part that allows the catheter to be connected to a syringe, a powered injector or other type of pump from which the contrast fluid to be injected is received. It also enables the clinician to navigate or maneuver the catheter, often with the aid of a guidewire, through the vasculature to the particular location (e.g., coronary artery or left ventricle) where the fluid is to be delivered. The strain relief is an intermediate section that provides a structural transition from the rigidity of the hub to the flexibility of the shaft. It prevents kinking of the catheter during handling and is color-coded for easy identification of the catheter's size. The shaft is the most predominant element of the catheter in that it constitutes a majority of its length. It is typically a composite of wire braiding sandwiched between two layers of plastic. This construction gives a catheter its ability to be pushed, pulled, twisted, and otherwise manipulated via the hub. The stem is generally a homogeneous plastic that is bonded to the shaft, and it is often shaped to allow catheterization of different arterial locations. The tip, at the distal end of the catheter, is a soft elastomeric material (e.g., plastic) that provides a cushion to prevent injury to the walls of the vasculature during the interventional procedure. Due to its tubular shape, the catheter defines a passage throughout its length, and this passage often includes an opening or endhole formed in the distal tip. Referred to as a lumen, this passage is the conduit through which fluid flows from the hub (into which the fluid is injected) to and out of the opening in the distal tip.

Diagnostic catheterization is a procedure that involves insertion of a catheter into an artery and guiding it to the desired location. The catheter can then be used to inject radiopaque dye, for example, with the aid of a manually-operated or automated pump. Using X-ray imaging techniques, the dye can be readily observed as it flows through the artery and any downstream branches, thereby providing the clinician with visual evidence of their condition and their ability to carry blood, usually to vital organs such as the heart, brain, kidney, etc. The major arteries of human body are shown in FIG. 2A, and those of the heart are shown in FIG. 2B. Coronary angiograms image the right and left coronary arteries, and ventriculograms are performed to evaluate the function of left and right ventricles. Aortograms are sometimes performed to obtain images of the ascending aorta and the aortic arch. Peripheral/radiology angiograms are typically performed on the carotid, cerebral, renal, femoral and popliteal arteries.

An example of how an infusion catheter can be used in a minimally invasive way to access the heart is shown in FIGS. 3A and 3B. A cardiac diagnostic catheterization procedure typically starts with a puncture into the femoral artery using a Seldinger needle. Once access is gained, a guidewire (typically 0.03 inches in diameter) is placed through the center of the needle into the artery, and the needle removed. A sheath with dilator of a given size, commonly called a vascular introducer, is then placed over the guidewire into the artery to expand the puncture site. The dilator and guidewire are then removed, leaving only the introducer with a hemostasis valve to seal against blood flow but allow access to the artery. The catheter and its associated guidewire are then inserted through the introducer into the artery, with the guidewire extending slightly beyond the tip of the catheter so that it protects the artery from puncture by the catheter as the catheter is routed into and through the vascular system. The guidewire and the tip of the catheter are radiopaque so that they can be observed via a fluoroscope as they are being guided to the targeted chamber or coronary artery. For the imaging of an artery, once the tip nears the artery to be imaged, the guidewire is then removed and the hub manipulated to place the tip of the catheter in the ostium (i.e., entrance) of the targeted coronary artery. Pressure within the artery is then measured to insure that the tip is placed appropriately (e.g., not embedded into a vessel wall) and the fluid path is unobstructed. Once the tip is securely positioned, a syringe is connected to the hub and the radiopaque fluid it contains is then injected into the catheter either manually or with a powered injector. Forced under pressure through the lumen of the catheter then out of the opening in its distal tip and, in some catheters, out of sideholes punched into the circular wall of its stem near the distal end, the contrast fluid then flows into the targeted artery. This procedure is repeated for each artery or chamber to be imaged. Upon completion of a catheterization, the catheter is removed from the introducer and the introducer is removed from the vascular system. The puncture wound is then sealed.

Cardiac catheterization is the thus process of inserting a catheter into an artery or vein, and routing it through that vessel and ultimately into the various vascular structures of the heart. It is used in measuring the pressure and flow of blood in the heart and its various blood vessels, in the diagnosis of congenital heart disease, and in exploring narrowed passages and other abnormal conditions. The catheter is routed to the heart typically with the aid of a fluoroscope or similar instrument, which displays real-time video images of the catheter as it is snaked through the vascular system to the desired site. More specifically, right heart catheterization involves insertion of a catheter into the femoral or subclavian veins for the purposes of: measuring pressure within the right atrium, right ventricle, or pulmonary artery; determining the degree to which oxygen is bound to hemoglobin in the blood (i.e., oxygen saturation); and ascertaining overall cardiac output. Left heart catheterization involves insertion of a catheter into the femoral or brachial arteries and then routing the catheter to the left side of the heart. It is used for the purposes of: determining whether there is stenosis (narrowing or constriction) of or regurgitation from the aortic valve (which normally prevents blood pumped into the aorta from flowing back into the left ventricle) or the mitral valve (which regulates the blood flow between the left atrium and left ventricle); ascertaining the global and regional functions of the left ventricle; and/or enabling images to be taken of the coronary arteries (ateriography) in conjunction with various imaging techniques.

Medical catheters are also used for a variety of purposes other than cardiac catheterization. It is well known that catheters can be used to deliver therapeutic drugs into vessels of the vascular system. For example, patients who have developed thrombolyses (i.e., clots) within a blood vessel are often candidates for catheterization. Clots are often manifested as soft or jelly-like clumps of blood or other cells, and they often, end up blocking a vein at a venous valve or an artery in a section thereof that is partially narrowed and sclerosed (i.e., hardened or thickened). However or wherever it forms, a clot that is dislodged and then carried from the place (e.g., vein, artery or chamber) where it formed to another location in the vasculature is called an embolus, and the resulting disorder is called an embolism. When a thrombus or embolism occurs in a vessel in the leg, for example, the afflicted patient will experience symptoms such as pain and loss of circulation. When occurring in a vessel of the lung (e.g., a pulmonary embolism), it can cause symptoms such as coughing, shortness of breath, chest pain, rapid breathing, and rapid heart rate (i.e., tachycardia). When a thrombus or embolism occurs in an artery of the brain, a stroke (i.e., an interruption in the supply of blood) occurs in the part of the brain supplied by that artery. Depending on the duration of the interruption and the part of the brain affected, a stroke can cause symptoms such as numbness, tingling or decreased sensation; vision problems; vertigo; difficulty in reading; inability to speak or to understand speech; loss of balance; paralysis of an arm, leg, side of the face, or other body part; loss of consciousness; and even death. In such circumstances, and often in lieu of surgery, thrombolytic agents are administered to break up blood clots and thus to restore the flow of blood to the affected area. Examples of thrombolytic agents include streptokinase, urokinase, and tissue plasminogen activator (TPA), and these agents are often delivered via an infusion catheter directly to affected portion of artery or vein where such clot-dissolving agents have best effect.

Smaller catheters are required for certain catheterization procedures, and would be preferred in others if not for heretofore unsolved problems. First, smaller diameter catheters require smaller incisions for insertion than do larger catheters such as the 5 or 6 French catheters used for cardiac catheterizations. Smaller incisions inflict less trauma upon patients, and thus require less labor to close and less time to heal as well as result in shorter hospital stays. Second, smaller catheters are significantly easier to navigate through narrower vessels. In any given catheterization procedure, there is a highly branched vessel network between the site of the incision and the targeted vessel, and the lumen of the vessel path leading from the insertion site to the targeted location typically becomes progressively smaller in diameter. The vessel path through which a catheter must be pushed and guided is therefore often narrow and tortuous, a task for which smaller catheters are better suited.

This is particularly true for catheters used in neurovascular applications. Blood vessels in the brain are as small as several millimeters or less in diameter, which require that catheters as small as 1 French be used. In addition to the small size of its vessels, the vasculature of the brain is highly branched and tortuous, requiring neurological catheters to be very flexible, especially at the distal ends, to pass through regions of such tortuosity. The vessels of the brain are quite delicate, so it is desirable for a catheter to have a soft, non-traumatic exterior surface and tip to prevent injury as noted above. Microcatheters, as such small diameter catheters are often called, are also capable of being snaked through the small branching arteries of other organs such as the liver.

A smaller diameter catheter, though, must be used with a powered injector rather than a manually-operated syringe to compel viscous fluid through its relatively small lumen. Only a powered injector can achieve and maintain the higher flow rate required for cardiac angiography, for example, when using such small catheters. This is because the same volume of contrast fluid must be delivered to the targeted artery for adequate imaging regardless of catheter size. As a result of this requirement, smaller diameter catheters pose certain disadvantages, namely the problems of “recoil” and “whipping.” These shortcomings are found not only in catheters in which the opening in the distal tip is the sole exit for the fluid, but also in catheters that have sideholes in the wall of the distal portion of the stem whether or not they have an opening in the distal end.

More specifically, certain catheter designs are known to give rise to fluidic forces that can cause the tip of the catheter to move as a result of the high velocity at which the fluid is ejected from the distal end. This unwanted tip motion is called “whipping” if it occurs in the plane of the tip and “recoil” if it occurs axially along the catheter. For example, in coronary catheterizations, the tip of the catheter can jump out of, or whip around, the ostium of a coronary artery due to the force with which the contrast fluid is pushed out the tip. Even larger diameter catheters will exhibit recoil and whip if the flow of fluid out of the distal end is of sufficient velocity. Much of the fluid will then miss the targeted artery and flow elsewhere downstream, resulting in wasted contrast fluid and unnecessary expense. Even more ominously, the high velocity of the misdirected fluid—and any whipping of the tip itself—can cause dissection of the vessel walls and dislodgement of plaque that may have accumulated there.

One way to reduce unwanted movement of the tip is to equip the catheter with peripheral sideholes, which act to reduce the amount of fluid that exits the distal opening. Usually a diverting means such as a valve or, more commonly, a restrictor is incorporated into the distal end of a catheter as one way to increase fluid pressure in the distal tip and thus encourage some of the fluid to flow out the sideholes. If the sideholes are not uniformly spaced about the circumference of the catheter, then the tip of the catheter will have a tendency to whip. Moreover, flow through the sideholes alone will not be sufficient to prevent recoil of a catheter during an injection. Any fluid flow out of the distal end of the catheter will produce a reaction that wants to forcibly push the tip backward out of the vessel, i.e., recoil. To minimize this force, either the flow out of the distal endhole must be reduced to a very small percentage of the total flow or eliminated entirely. Alternatively, angled sideholes could be used to provide a counterbalancing hydraulic force to that created by the fluid flowing out of the distal endhole.

Another problem with various prior art catheters involves streaming effects. This is the tendency of the contrast fluid upon exit from the tip of the catheter to remain concentrated, i.e., the fluid will not be widely and finely dispersed within the targeted area When this occurs, the targeted vessel has not received optimal opacification (i.e., rendering the targeted vessel readily discernable via imaging equipment) and thus the flow of fluid therethrough cannot be well observed during the imaging procedure.

Several prior art patents disclose catheters that exhibit disadvantages the same as, or even beyond those, mentioned above. U.S. Pat. No. 3,828,767 to Spiroff discloses a catheter design in which fluidic forces are purportedly balanced both radially (via fluid flowing out of large sideholes in the wall of the catheter) and axially (via fluid flowing out of an opening in the distal end opposed by fluid flowing out of proximally-angled sideholes in the cylindrical wall). Regardless of how well the Spiroff design balances the radial and axial forces of injection/infusion, it still permits fluid to flow at high velocity out of the opening in the distal end of the catheter, which creates the potential for dissection of tissue and dislodgement of plaque from the vessel walls. It also reduces the amount of fluid that will flow out of the sideholes. Furthermore, the large diameter of the sideholes (as evidenced by the punching operation by which they are made) coupled with the large diameter of the distal opening prevents the fluid flowing therefrom from being very finely dispersed about the porous tip of the Spiroff catheter.

U.S. Pat. No. 5,843,050 to Jones et al. discloses several microcatheter designs. The microcatheter shown in FIG. 6 of that reference features a valve in its distal end that allows passage of a guidewire. Because the valve is always open, however, this catheter is similar to the Spiroff design in that it permits fluid to flow at high velocity out of an aperture/endhole in its distal end. FIGS. 5 and 8-13, in contrast, each illustrate a microcatheter that has a normally-closed valve in its distal end. Although these valves allow passage of a guidewire, they do not permit measurement of the pressure within the vessel through the catheter. U.S. Pat. No. 5,085,635 to Cragg also discloses a normally-closed valve over the distal endhole of the catheter, along with relatively large sideholes about its distal end from which fluid is discharged laterally. Although the leaflet-type valve taught by Cragg allows passage of a guidewire, it effectively blocks flow of fluid through the endhole and thus completely prevents hemodynamic measurements.

U.S. Pat. No. 6,669,679 to Savage et al., and its corresponding WIPO Publication WO/0151116, disclose a catheter having a small number of sideholes angled in the proximal direction along with an elastic opening in its distal end that allows passage of a guidewire. The sideholes are made via a punching process, which is responsible for their large diameter (0.254 mm and larger). Quite similar to the disclosure in the Spiroff patent, the '679 patent claims use of a catheter that balances the forces acting upon it by (i) “variably restricting” the flow of fluid through the opening in the distal end and (ii) directing fluid out of the proximally-angled sideholes in the wall of the catheter. This “variably restricting” function, however, is carried out solely by use of the elastic opening. And, due to its elasticity, this opening merely increases in diameter as the pressure of fluid increases within the catheter, thus permitting fluid to flow out the distal end at a relatively high velocity. This catheter thus poses a comparatively high risk of dissection of tissue and dislodgement of plaque from the vessel walls. Another shortcoming of this catheter design is that its large sideholes prevents the fluid from being finely dispersed from the distal end as compared to the invention disclosed below.

U.S. Pat. No. 5,807,349 to Person et al. discloses a catheter having a hinge-type valve over an opening in its distal end through which fluid can be infused into, or drawn from, a vessel in the body. The hinge flexes outwardly from the opening during infusion/injection, and flexes inwardly into the opening when fluid is being drawn into the catheter. Due to its ability to flex, this normally-closed hinge-type valve functions as a variable opening, as the degree of infusion or egress of fluid depends on the amount of pressure or vacuum that exists within the lumen of the catheter. Person et al. thus appears to teach a distal opening that is functionally identical to the elastic opening (i.e., “variable restrictor”) claimed by the '679 patent in that the “opening” of each merely increases in diameter as the pressure of fluid increases within the catheter. Together, the balanced fluidic forces taught by Spiroff and the variable opening taught by Person et al. appear to present the catheter, and the concomitant disadvantages, of the '679 patent.

There is therefore a need to develop a catheter that overcomes the disadvantages inherent to the prior art. In particular, it would be desirable to devise a catheter whose distal end remains exceptionally stable within the vasculature while fluid is being very finely dispersed therefrom during interventional procedures. It would also be advantageous if the fluid exiting the sideholes of the catheter could be far more finely dispersed about the perimeter of the stem than is possible with currently known devices. It would also be beneficial if the catheter could be equipped with an opening in its distal end out of which the fluid would exit at a velocity substantially lower than prior art designs. It would also be ideal if a catheter could be equipped with a restrictor in its distal end whose opening tended to decrease in size as pressure in the lumen increased, and did so such that the forces of fluid flowing out of the distal opening and out of the sideholes in the stem would be substantially in balance to prevent whipping and recoil while fluid is being finely dispersed therefrom in a cloud-like form.

### SUMMARY OF THE INVENTION

Several objectives and advantages of the invention are attained by the preferred and alternative embodiments and related aspects of the invention summarized below.

In one presently preferred embodiment, the invention provides a catheter assembly for introducing fluid into a vessel. The catheter assembly includes a shaft, a hub affixed to a proximal end of the shaft, a stem affixed to a distal end of the shaft, and a tip affixed to the distal end of the stem. The stem has a porous section approximate a distal end thereof. The porous section defines a plurality of microholes generally distributed uniformly thereabout and inclined by a predetermined angle in a proximal direction. The tip includes a conically-shaped valve with an apex thereof pointing in the proximal direction and defining an opening thereat. As the fluid flows within the catheter assembly and pressure increases within the tip, the conically-shaped valve tends to flatten out distally thereby generally decreasing a size of the opening so that the amount of the fluid flowing out of the opening of the tip decreases and that out of the microholes of the stem increases. The forces of the fluid flowing out of the microholes and the opening substantially balance thereby enabling the position of the tip and stem within the vessel to remain stable while fluid is finely dispersed therefrom.

In a related embodiment, the invention provides a catheter assembly for introducing fluid into a vessel. The catheter assembly includes a stem and a tip affixed to a distal end of the stem. The stem has approximate its distal end a porous section. The porous section defines a plurality of microholes distributed thereabout, which are inclined by a predetermined angle in a proximal direction. The tip includes a conically-shaped valve with an apex thereof pointed in the proximal direction. The apex defines an opening whose size generally decreases as the conically-shaped valve flattens out distally as pressure of the fluid within the tip increases. The forces of the fluid flowing from within the catheter assembly out of the opening of the tip and out of the microholes of the stem substantially balance thereby preventing both recoil and whipping of the catheter assembly thus enabling the position thereof within the vessel to remain stable while the fluid is finely dispersed therefrom.

In a related aspect, the invention provides a catheter assembly for introducing fluid into a vessel. The catheter assembly includes a restrictor at a distal end thereof. The restrictor includes a conically-shaped valve comprising a circular base portion and a conical wall portion. The circular base portion is formed approximate a distal end of the restrictor. The conical wall portion extends in a proximal direction from the circular base portion to an apex thereof. The apex defines an opening whose size generally decreases as the conically-shaped valve flattens out distally as pressure of the fluid within the restrictor increases.

In related embodiment, the invention provides a catheter assembly for introducing fluid into a vessel. The catheter assembly includes a stem and a restrictor affixed to a distal end of the stem. The stem has approximate its distal end a porous section. The porous section defines a plurality of microholes distributed thereabout, which are inclined by a predetermined angle in a proximal direction. The restrictor defines an opening therein whose size generally decreases as pressure of the fluid within the restrictor increases. The forces of the fluid flowing from within the catheter assembly out of the opening of the restrictor and out of the microholes of the stem substantially balance to prevent axial and radial movement of the catheter assembly thus enabling a position thereof within the vessel to remain stable while the fluid is finely dispersed therefrom in a cloud-like form.

In a related aspect, the invention provides a catheter comprising a distal segment. The distal segment includes a porous section and a restrictor. The restrictor is contiguous with the porous section and defines an opening therein whose size generally decreases as pressure of fluid within the restrictor increases.

In a related aspect, the invention provides a catheter comprising a restrictor approximate its distal end. The restrictor defines an opening therein whose size generally decreases as pressure of fluid within the restrictor increases.

In another related aspect, the invention provides a catheter that includes a shaft and a stem. Affixed to the distal end of the shaft, the stem has a porous section that defines a plurality of microholes.

In broader application, the invention provides an injector system. The injector system comprises an injector and a catheter. The injector is used for injecting a fluid into a patient. The catheter is operably associated with the injector for introducing the fluid into a bodily structure. The catheter comprises a porous section and a restrictor contiguous with the porous section. The restrictor defines an opening therein whose size generally decreases as pressure of fluid within the restrictor increases.

### BRIEF DESCRIPTION OF THE DRAWINGS

The invention, and particularly its presently preferred and alternative embodiments and related aspects, will be better understood by reference to the detailed disclosure below and to the accompanying drawings, in which:

FIG. 1A illustrates the basic construction of a prior art catheter assembly inclusive of the hub, the strain relief element, the shaft, the stem, and the tip.

FIG. 1B illustrates a Judkins catheter, which is a selective coronary artery catheter available in different shapes for catheterizations of the left and right coronary arteries.

FIG. 1C illustrates a Amplatz catheter, which is a selective coronary artery catheter available in different shapes for catheterizations of the left and right coronary arteries.

FIG. 1D illustrates a Coronary Bypass catheter, which is a selective catheter available in different shapes for catheterizations involving the left and right coronary arteries.

FIG. 1E illustrates a Pigtail catheter, which is a flush catheter available in different shapes and typically used for catheterizations of the ventricles and the aorta.

FIGS. 2A and 2B illustrate the major arteries of the human body and the heart.

FIGS. 3A and 3B illustrate the minimally invasive way that a catheter can provide access to a targeted vessel or organ within the human body.

FIG. 4A illustrates the distal segment of a catheter constructed according to a first embodiment of the invention.

FIG. 4B illustrates a cross-sectional schematic view of the catheter shown in FIG. 4A in the context of a 4 French catheter, showing the novel restrictor and the microholes of the porous section inclined by a predetermined angle in the proximal direction.

FIG. 4C illustrates an enlarged view of a portion of the catheter shown in FIG. 4B, showing the region where the novel restrictor and porous section of the stem interface.

FIG. 4D illustrates an “unwrapped” view of the porous section of the catheter shown in FIG. 4B, showing its preferred microhole pattern.

FIG. 4E illustrates an enlarged view of a portion of the microhole pattern shown in FIG. 4D.

FIG. 4F illustrates an “unwrapped” view of the porous section of the catheter shown in FIG. 4B, but in which an alternative microhole pattern has been implemented.

FIG. 4G illustrates a cross-sectional schematic view of the catheter shown in FIG. 4A in the context of a 5 French catheter, showing the novel restrictor and the microholes of the porous section inclined by a predetermined angle in the proximal direction.

FIG. 4H illustrates an enlarged view of a portion of the catheter shown in FIG. 4G, showing the region where the novel restrictor and porous section of the stem interface.

FIG. 41 illustrates an “unwrapped” view of the porous section of the catheter shown in FIG. 4G, in which the preferred microhole pattern has been implemented.

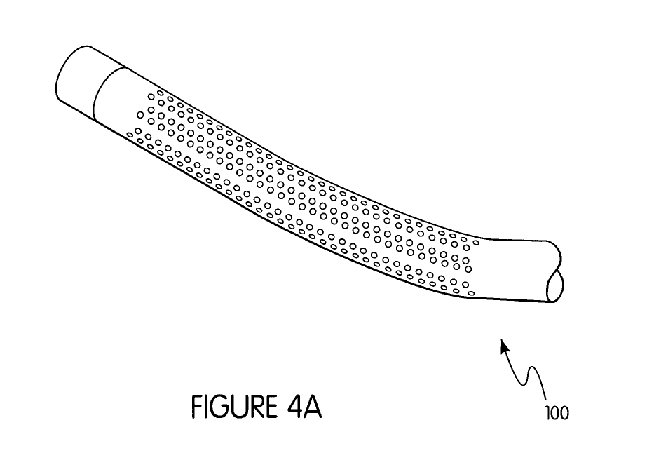

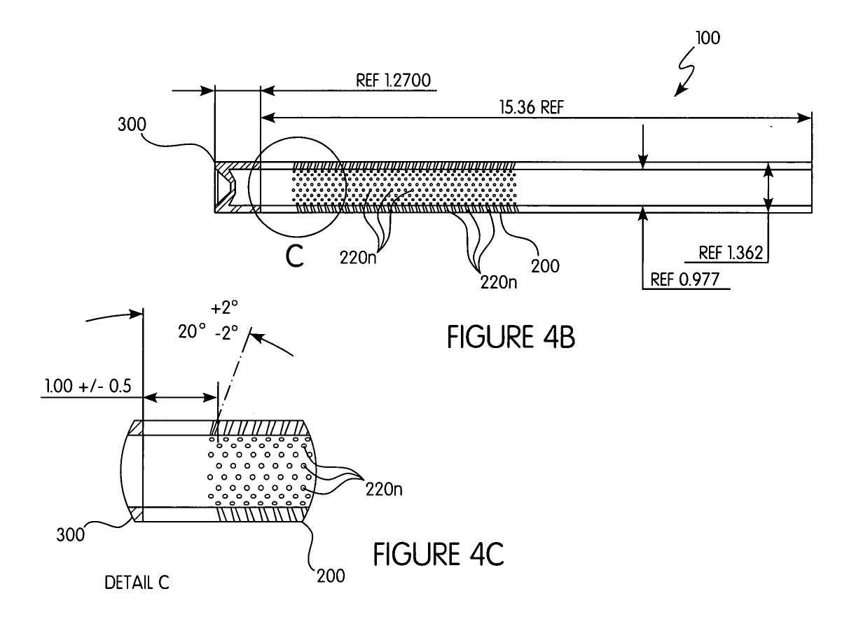

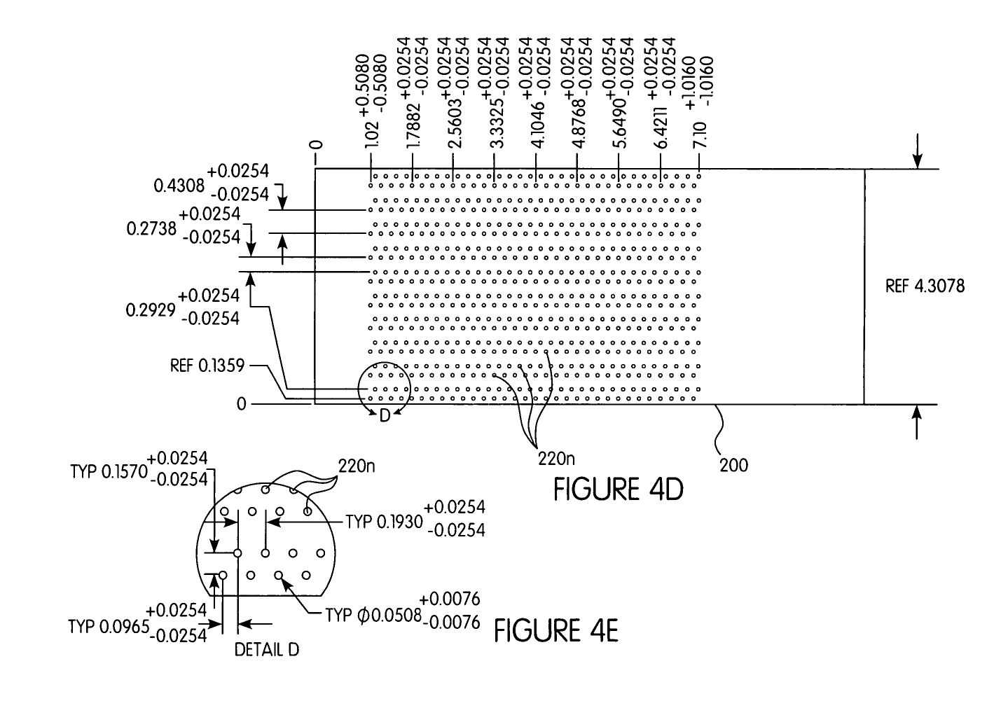

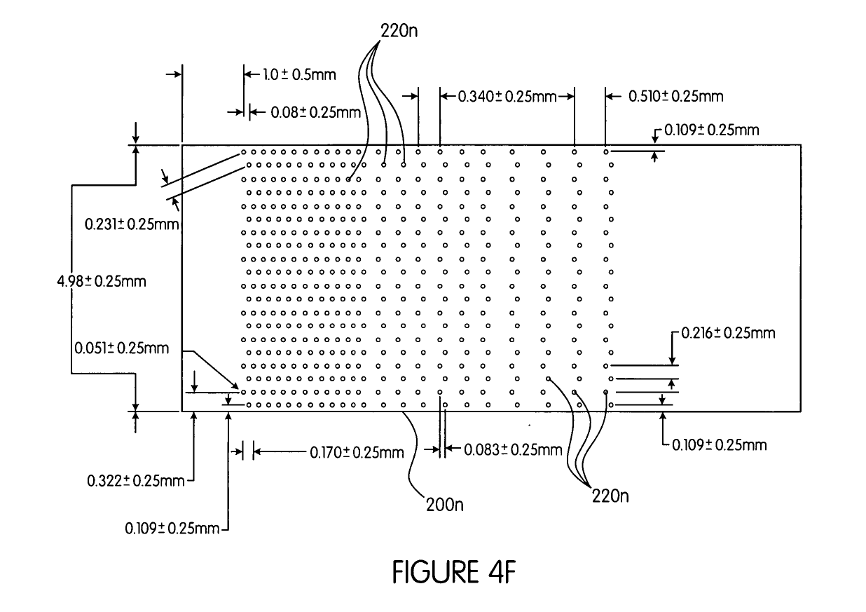

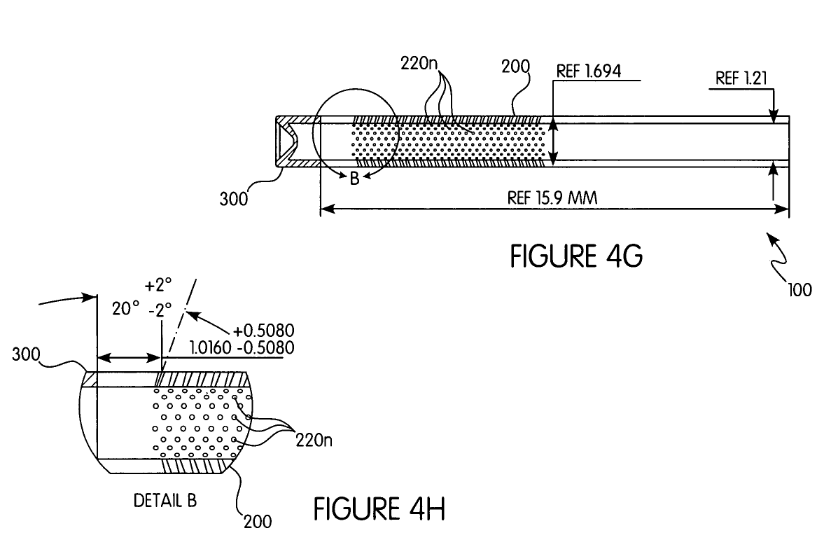

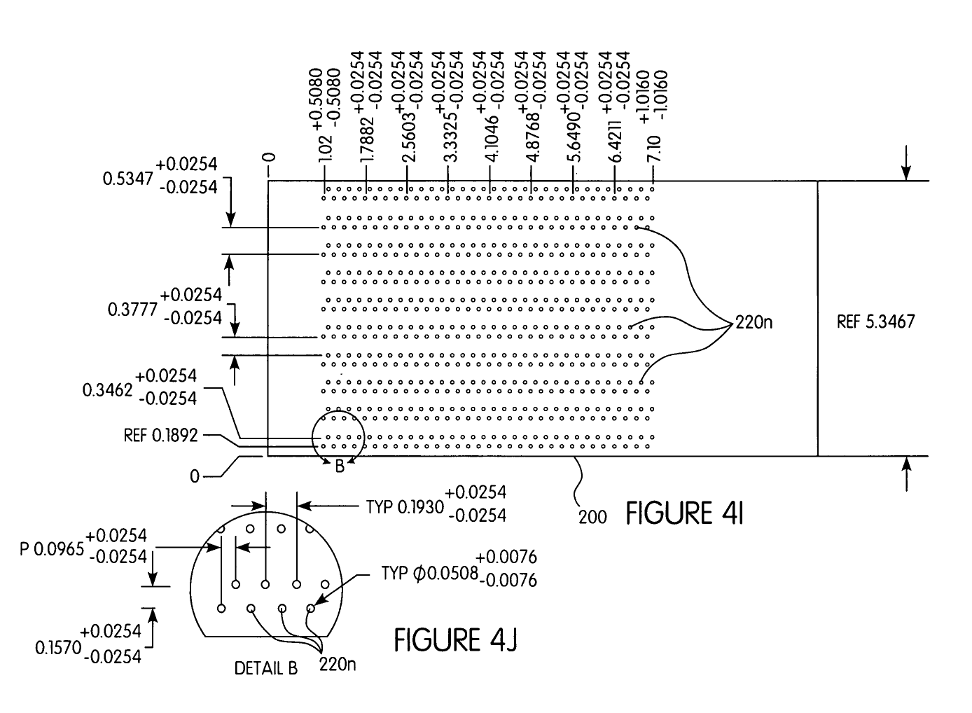

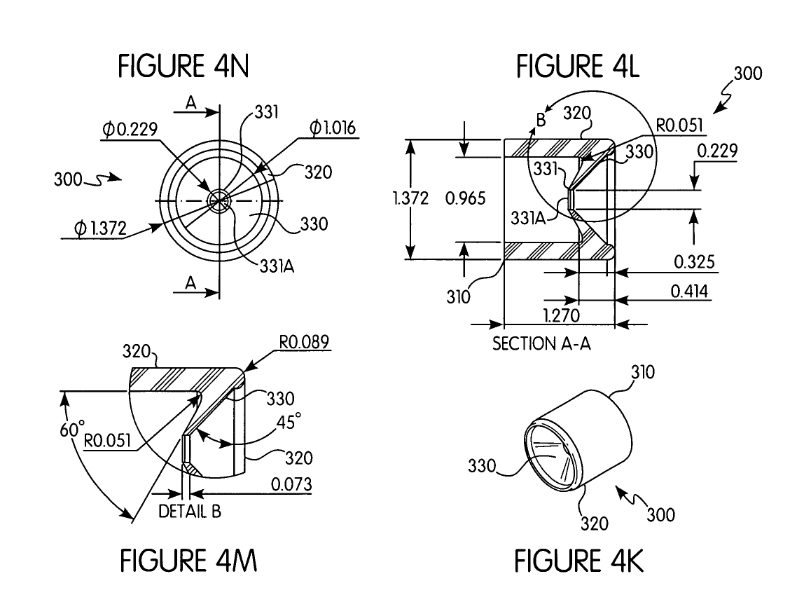

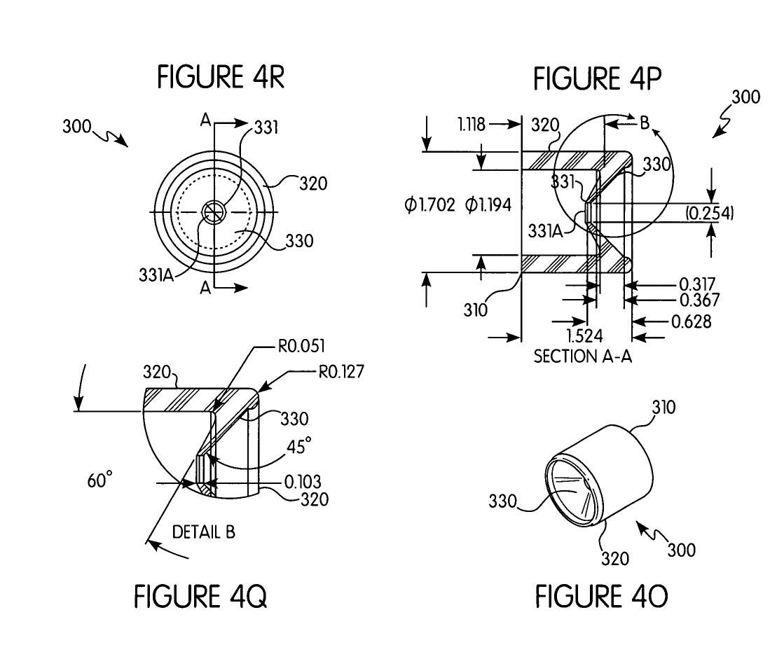

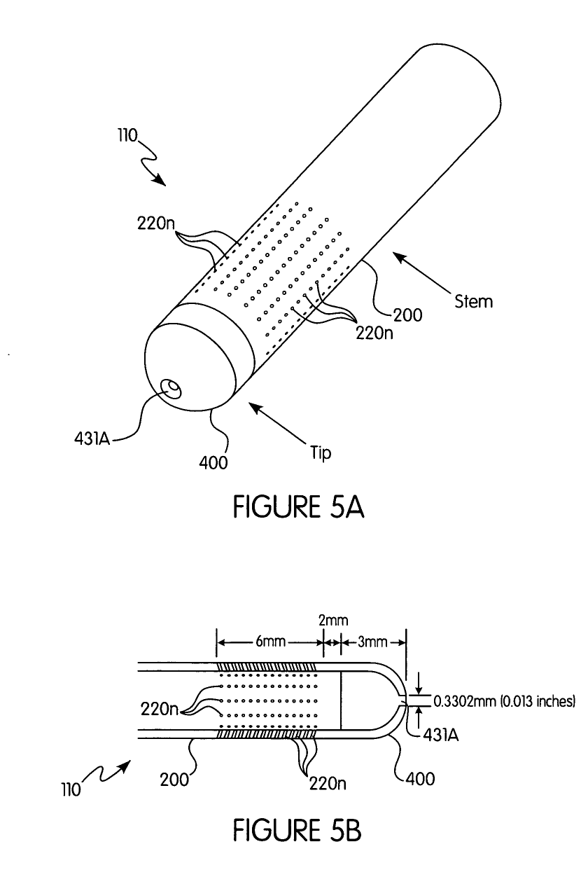

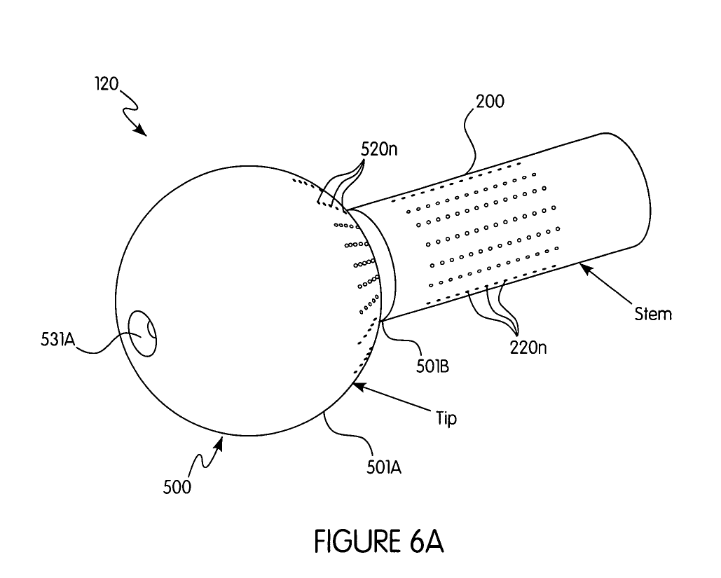

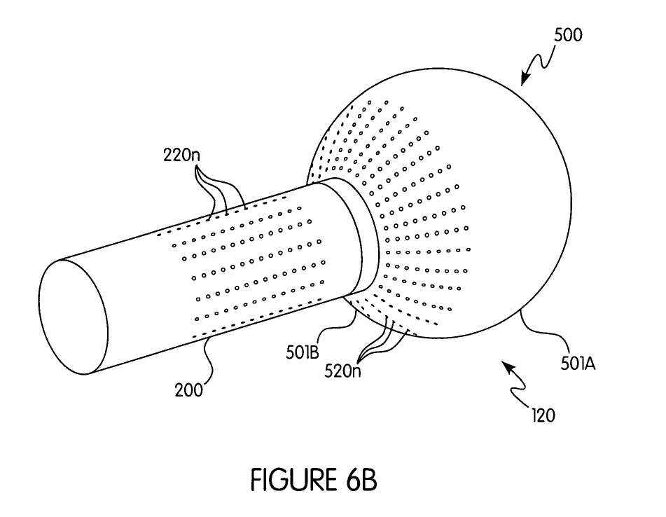

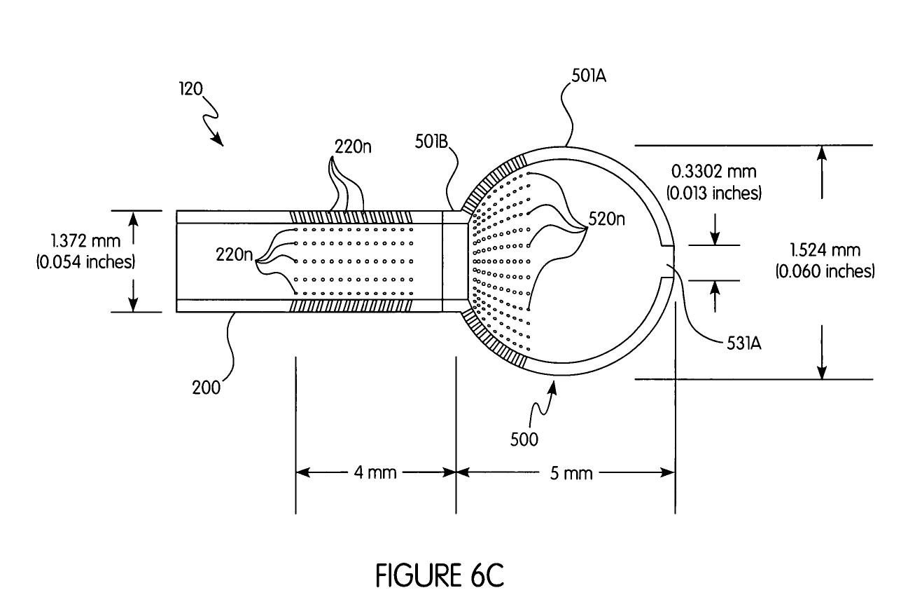

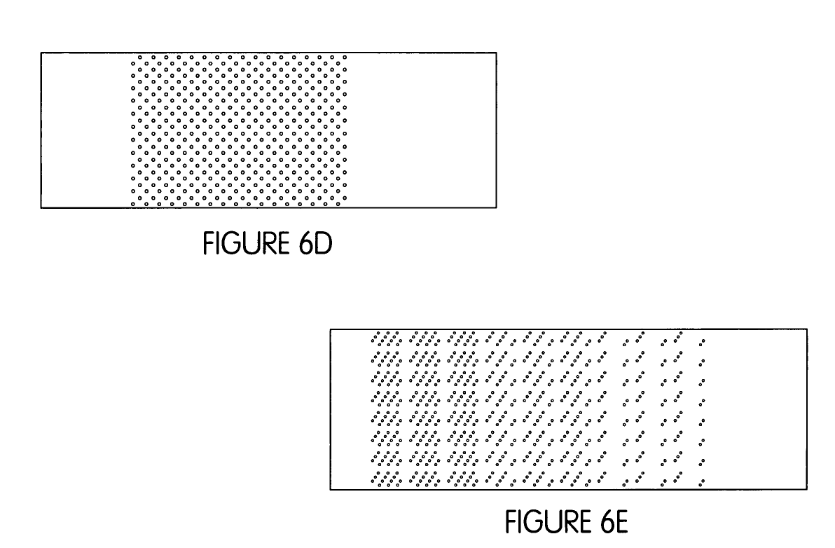

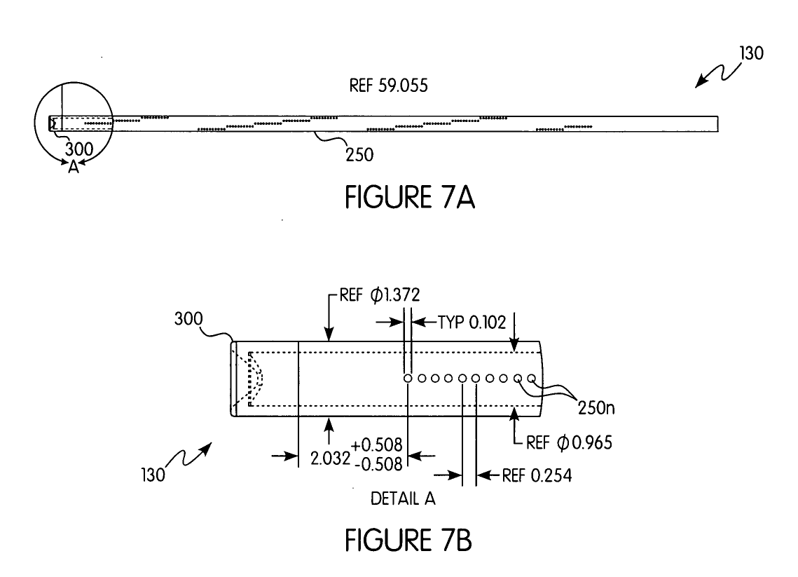

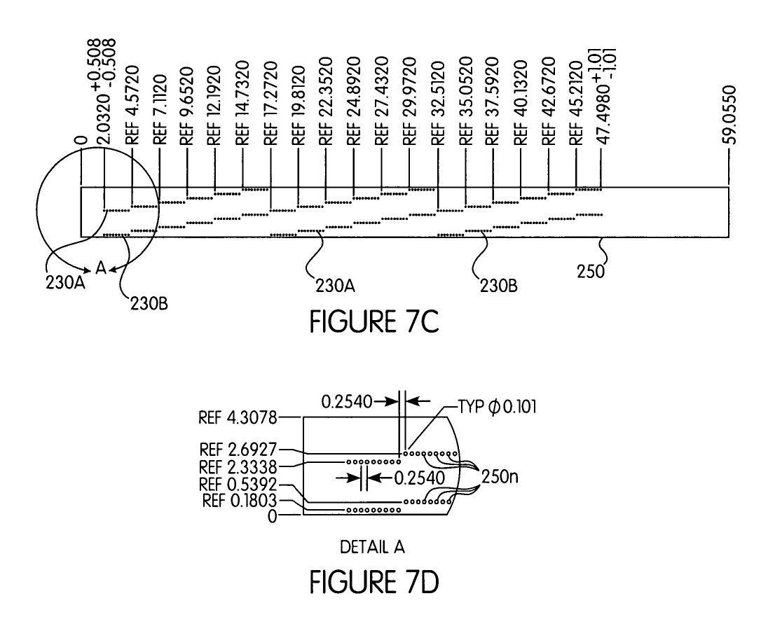

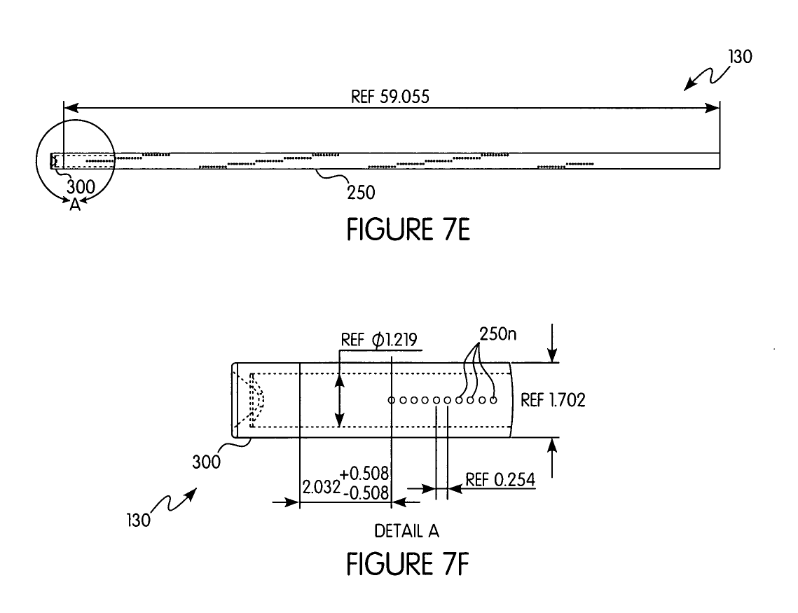

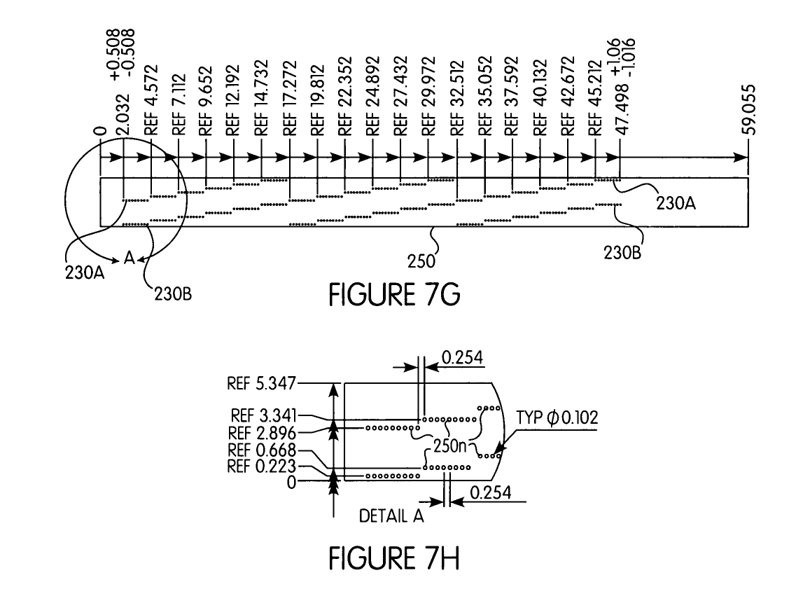

FIG. 4J illustrates an enlarged view of a portion of the microhole pattern shown in FIG. 4I.

FIGS. 4K-4N illustrate a preferred manifestation of the restrictor for the 4 French catheter shown in FIG. 4B.

FIGS. 4O-4R illustrate a preferred manifestation of the restrictor for the 5 French catheter shown in FIG. 4G.

FIG. 5A illustrates a perspective view of the distal segment of a catheter constructed according to a second embodiment of the invention.

FIG. 5B illustrates an enlarged cross-sectional view of the catheter shown in FIG. 5A, showing an alternative restrictor and the microholes of the porous section inclined by a predetermined angle in the proximal direction.

FIG. 6A illustrates in perspective the distal segment of a catheter constructed according to a third embodiment of the invention.

FIG. 6B is a perspective view of the catheter of FIG. 6A as viewed from the opposite end.

FIG. 6C illustrates an enlarged cross-sectional view of the catheter of FIGS. 6A and 6B, showing a different type of restrictor and the microholes of the porous section inclined by a predetermined angle in the proximal direction.

FIG. 6D illustrates one type of microhole pattern, in which the microholes are uniformly distributed about the porous section, that can be implemented on the catheter of FIGS. 6A-6C.

FIG. 6E illustrates an alternative microhole pattern, in which the microholes are deployed according to a gradient in the longitudinal direction in three sections of approximately equal length, that can be implemented on the catheter of FIGS. 6A-6C.

FIG. 7A illustrates a side view of the distal segment of a catheter constructed according to a fourth embodiment of the invention in the context of a 4 French catheter.

FIG. 7B illustrates an enlarged cross-sectional view of a portion of the catheter shown in FIG. 7A, showing the region where the novel restrictor and porous section of the stem interface.

FIG. 7C illustrates an “unwrapped” view of the porous section of the catheter shown in FIG. 7A, showing its preferred microhole pattern.

FIG. 7D illustrates an enlarged view of a portion of the microhole pattern shown in FIG. 7C.

FIG. 7E illustrates a side view of a catheter constructed according to the fourth embodiment of the invention, but in the context of a 5 French catheter.

FIG. 7F illustrates an enlarged cross-sectional view of a portion of the catheter shown in FIG. 7E, showing the region where the novel restrictor and porous section of the stem interface.

FIG. 7G illustrates an “unwrapped” view of the porous section of the catheter shown in FIG. 7E, showing its preferred microhole pattern.

FIG. 7H illustrates an enlarged view of a portion of the microhole pattern shown in FIG. 7G.

### DETAILED DESCRIPTION OF THE INVENTION

FIGS. 4A-7H illustrate several embodiments and various preferred and optional aspects of the invention, namely, a catheter capable of being used for diagnostic imaging, therapeutic treatments and various other diagnostic and interventional procedures. Like many of the prior art catheters noted in background, the catheter assemblies of the present invention will also generally include a hub, a strain relief section, a shaft, and a stem. Although the invention herein described and illustrated is presented primarily in the context of cardiac or angiographic catheters, the reader should understand that it can be applied or adapted to catheters of widely different types, shapes, sizes and purposes.

FIGS. 4A-4R illustrate a first embodiment of the invention along with various preferred and alternative aspects. The catheter, generally designated 100, includes a stem equipped with a porous section 200 and a restrictor 300 affixed to the distal end of the stem. As shown in FIG. 4B, the stem is approximately 15.36 mm in length for catheter 100 made in a 4 French size, with approximate outer and inner (lumen) diameters of 1.362 mm and 0.977 mm, respectively. For catheter 100 in the 5 French size shown in FIG. 4G, the stem has a length of approximately 15.9 mm, with approximate outer and inner diameters of 1.694 mm and 1.21 mm.

Preferably located in proximity to the distal end of the stem, the porous section 200 includes a large plurality of microholes 220n, each of which in communication with the lumen of the catheter 100. For reasons explained in more detail below, all microholes 220n in the porous section 200 are preferably made having the same diameter. Although the diameter is generally best set between approximately 5 to 250 microns, the preferred diameter for the microholes 220n is about 50 microns in this embodiment. This diameter is shown in FIGS. 4E and 4J as being 0.0508±0.0076 mm for catheters 100 of 4 French and 5 French size, respectively.

The microholes in this first embodiment are also inclined by a predetermined angle in the proximal direction with respect to a plane normal to the longitudinal axis of catheter 100. This predetermined angle preferably ranges approximately from 0 to 45 degrees, with the exact angle being dependent upon several factors such as the size, length, and shape of the catheter and the volume of fluid injected therethrough; the size, location and deployment of the microholes; the manner in which the restrictor of the invention is to be implemented, and the ratio of the amount of fluid to be flowing out of the microholes 220n to that flowing out of a distal endhole, if any. In the first embodiment disclosed herein, the predetermined angle is best set at approximately 20 degrees. This angle is shown in FIGS. 4B-4C and 4G-4H as being 20±2 degrees for catheters 100 of 4 French and 5 French size, respectively.

Whether positioned near the distal end of the stem or elsewhere along its length, the microholes may be deployed according to any one or more of a variety of patterns. Although two patterns are disclosed below in connection with the first embodiment of the present catheter, it should be apparent that other patterns could also be employed.

FIGS. 4B and 4G illustrate the microholes 220n deployed near the distal end according to a preferred pattern in which they are uniformly distributed both longitudinally along the axis of the catheter and radially about its circumference. FIGS. 4D and 4I show the preferred microhole pattern in greater detail for catheter 100 in a 4 French size and a 5 French size, respectively. In these views, the porous section 200 of catheter 100 has been unwrapped to a flat sheet from its normal cylindrical shape. FIG. 4D shows the circumference for a 4 French catheter to be approximately 4.3078 mm, and FIG. 4I shows a circumference of 5.3467 mm for a 5 French catheter 100.

FIGS. 4D and 4I show that the preferred microhole pattern takes the form of 10 pairs of longitudinally arranged rows. In each row pair, as best shown in FIGS. 4E and 4J, the rows are spaced laterally by 0.1570±0.0254 mm, with one row offset longitudinally from the other by 0.0965±0.0254 mm. The microholes 220n in each row are separated longitudinally by 0.1930±0.0254 mm. The row pairs are spaced laterally by 0.2738±0.0254 mm for the 4 French catheter 100 shown in FIG. 4D, and by 0.3777±0.0254 mm for the 5 French catheter 100 shown in FIG. 4I.

The preferred length of porous section 200 is approximately 6 mm, and the number of microholes 220n it contains is preferably about n=640. This is best shown in FIGS. 4D and 4I for the 4 and 5 French size catheters 100, respectively. In addition, porous section 200 is preferably spaced approximately 1 mm from the distal end of the stem to which the restrictor 300 is to be affixed. This distance is shown in FIGS. 4C and 4H as being 1.00±0.5 mm and 1.0160±0.5080 mm, respectively, for catheters 100 of 4 and 5 French size, respectively. This particular pattern is well suited for catheter applications in which the dispersion of fluid is to be confined to a relatively small region of the catheter, e.g., near its distal tip. Such a confined, fine dispersion of fluid is, for example, ideal for injection of contrast fluid into the ostium of a coronary artery, as it enables the fluid to be carried therein by the flow of blood.

FIG. 4F illustrates an alternative pattern for the microholes 220n, one which is divided into three substantially equal sections along the length of the porous region 200. The most proximal section contains the least number of microholes, which can be defined numerically as X. The second section contains twice as many microholes, i.e., 2×, as the first section. The third section contains three times as many microholes, i.e., 3×, as the first section. In each of the three sections, the microholes are deployed longitudinally in rows, with each row being spaced 0.216±0.25 mm (0.0085±0.0010 in) from its neighbor and every other row being offset in the proximal direction by about 0.083±0.25 mm (0.0033±0.0010 in). The microholes in each row are separated by 0.170±0.25 mm (0.0067±0.0010 in) in the first section, 0.340±0.25 mm (0.0134±0.0010 in) in the second section, and 0.510±0.25 mm (0.0201±0.0010 in) in the third section. This particular pattern as illustrated contains n=440 microholes, with a diameter of 0.051±0.75 mm (0.0020±0.0003 in), and with either all or some (e.g., selected groups) of the microholes being inclined 20 degrees in the proximal direction. In addition, the porous section 200 is spaced 1.0±0.5 mm (0.040±0.020 in) from the distal end of the stem to which the restrictor or tip 300 is to be affixed. This spacing of porous section 200 is also appropriate for the preferred pattern of microholes 220n shown in FIGS. 4D and 4I. If this pattern were to be implemented on the catheter of the present invention, the increase in microhole density toward the tip would decrease the hydraulic resistance of the sideholes, which would offset the decreased pressure of the fluid as it flows axially through the lumen of the catheter. For certain applications, this would permit a more uniform distribution of fluid from the catheter. This pattern may be more desirable for injecting contrast fluids over longer lengths, e.g., 10 cm for catheters used in interventional procedures such as abdominal aortograms.

Another way to implement a change in hydraulic resistance along the length of a catheter is to use a uniform microhole pattern but change the diameter of the microholes. If this concept were to be implemented on the catheter(s) of the present invention, the increase in diameter of the microholes toward the tip would decrease the hydraulic resistance of the sideholes, which would offset the decreased pressure of the fluid as it flows axially through the lumen of the catheter. This alternative—i.e., the diameter of the microholes of the porous section changing with position along the stem—may be implemented on any of the disclosed embodiments.

The choice of microhole pattern will, of course, generally depend upon which of the novel restrictors disclosed below is selected for incorporation as part of the catheter. For the preferred embodiment of the invention, the microhole pattern may require either all or some of the microholes to be inclined in the proximal direction. How many of the microholes are to be inclined—and, as noted above, the angle(s) of inclination—depends on a number of factors such as the size, length, and shape of the catheter and the volume of fluid injected therethrough; the size, location and deployment of the microholes; and the ratio of the amount of fluid that one wants to flow out of the microholes versus that, if any, out of the restrictor. Regardless of their number or inclination, the microholes will still have to be distributed circumferentially in such a way as to avoid whipping of the resulting catheter.

The restrictor 300 in this first embodiment takes the form of a conically-shaped valve 310 whose apex 331 points in the proximal direction. This is shown in FIGS. 4K-4N and 4O-4R for catheter 100 in the context of 4 and 5 French sizes, respectively. As best shown in FIGS. 4K & 4L and 40 & 4P, the conically-shaped valve 310 includes a circular base portion 320 and a conical wall portion 330. The circular base portion 320 is bonded or otherwise affixed to the distal end of the stem, as is shown in FIGS. 4B and 4G. The conical wall portion 330 extends and decreases in thickness from the distal end of circular base portion 320 to the apex 331, as is best illustrated in FIGS. 4L & 4M and 4P & 4Q. The region over which the conical wall portion 330 attaches to or emerges from circular base portion 320 tends to act like a hinge as will become apparent below. As best shown in FIGS. 4L-4N and 4P-4R, the apex 331 features or is otherwise truncated to define an opening 331A at the proximal end of the valve.

FIGS. 4K-4N illustrate the restrictor 300 for a 4 French catheter 100, with its circular base portion 320 having an outer diameter of 1.372 mm and an inner diameter of 0.965 mm. FIGS. 4O-4R show these outer and inner diameters as 1.702 mm and 1.194 mm, respectively, for restrictor 300 of a 5 French catheter 100. In its preferred manifestation, the conical wall portion 330 on its proximal surface forms an angle of 60 degrees with the inner wall of circular base portion 320, as shown in FIGS. 4M and 4Q. On its distal surface, however, the conical wall portion 330 forms an angle of 45 degrees with that wall. The differing angles of the proximal and distal surfaces of conical wall portion 330 are thus responsible for the decreasing thickness of conical wall portion 330 as it extends proximally from the distal end of circular base portion 320. Although the length of tip 300 preferably ranges from 1 to 10 mm or even longer, it is preferably 1.270 mm and 1.524 mm for a catheter 100 of 4 and 5 French sizes, respectively, as best shown in FIGS. 4L and 4P.

The opening 331A of tip 300 permits passage of a guidewire to facilitate insertion of catheter 100 into the body and the routing of its distal end to the targeted vessel, chamber or cavity. Although smaller than the lumen of the stem, the size of the opening 331A is able to expand to fit guidewires of slightly larger diameter due its elasticity. The conically-shaped valve 310 is preferably constructed so that the diameter of its opening 331A at the apex 331 is approximately 0.229 mm and 0.254 mm for 4 and 5 French size restrictors 300, as shown in FIGS. 4L and 4P, respectively. The diameter, however, may range generally from 0.1016 mm (0.004 inches) at the apex to 0.889 mm (0.035 inches) at the distal end of circular base portion 320 in this first embodiment. The conical wall portion 330 also dynamically changes its shape by moving distally and compressing radially inward as fluid flows into catheter 100 and the pressure on the proximal side of valve 310 increases above a design-dependent threshold. Consequently, the opening 331A in this preferred manifestation of restrictor 300 should accommodate changes in its diameter in the range of approximately 0.0762 to 0.127 mm (0.003 to 0.005 inches) in response to such pressure changes. If larger or smaller decreases in the diameter of opening 331A are desired in response to such pressure changes, they can be achieved by changing the thickness, shape or composition of conical wall portion 330 or other aspects of restrictor 300. The exact size of opening 331A will, of course, be dependent upon factors such as the size, length, and shape of catheter 100 and the volume of fluid injected therethrough; the size, location and deployment of the microholes 220n; and the ratio of the amount of fluid that one wants to flow out of the opening 331A versus that out of the microholes 220n.

In addition to functioning as a diverter to direct fluid out of the microholes, the restrictor(s) of the invention are preferably radiopaque so that they can be observed via a fluoroscope as the tip of the catheter is being guided to the targeted vessel or chamber.

Once catheter 100 is guided though the anatomy and its distal segment properly positioned at the desired location, the injector or other pump to which the hub is connected will be activated to pressurize the fluid to be administered. This causes the fluid to flow through the lumen of catheter 100 and ultimately to the tip 300. More specifically, the pump causes the fluid to flow into the hub, through the shaft and stem, and into the distal segment of the catheter 100. Once the fluid reaches the distal segment, fluid begins to flow out of opening 331A and pressure begins to build against the proximal side of conically shaped valve 310. Increased hydraulic pressure, however, will be required to push the fluid through the microholes 220n of porous section 200. As soon as the pressure reaches the design-dependent threshold, the conical wall portion 330 begins to flatten out distally thereby decreasing the size of opening 331A. In response to the decreasing size of opening 331A, the amount of the fluid flowing out of opening 331A decreases while the fluid flowing out microholes 220n of porous section 200 increases accordingly. From the opening 331A of restrictor 300 and, to a greater extent, the microholes 220n, the fluid then flows as a very fine, cloud-like dispersion into the targeted vessel, chamber or cavity.

Despite the high pressure extant within its lumen, this first embodiment of catheter 100 not only ensures the stability of its distal end but also discharges therefrom a very fine dispersion of the fluid at very low velocities. The stability of the catheter during an injection is achieved by balancing the fluidic forces both axially and radially. Recoil or axial movement of catheter 100 is avoided because the force of the fluid flowing out of opening 331A in the distal direction is substantially balanced by the cumulative force of the fluid flowing out of the inclined microholes 220n in the proximal direction. Whipping of catheter 100 is forestalled because the forces of the fluid flowing radially out of the porous section 200 are substantially balanced due to the uniform distribution of the microholes 220n about its circumference. Consequently, in coronary catheterizations, for example, the distal end of catheter 100 will remain exceptionally stable in the ostium of the coronary artery. The bolus of fluid emanating from the distal end will then flow into the targeted artery rather than be substantially misdirected as is typical with many of the catheters noted in background and others known in the art.

The pattern of dispersion provided by catheter 100 allows even the ostial region of a vessel to be imaged, a result which is not as feasible with prior art angiographic catheters. Ideally, catheter 100 can be configured so that 90% or more of the fluid is very finely dispensed through the microholes 220n in a cloud-like form, with the remainder exiting the distal opening 331A at a very low velocity. Alternatively, the percentage of the fluid flowing out of the microholes versus that out of the opening could be set at 51% to 49%, respectively, or even lower. Used with standard contrast media, catheter 100 has exhibited in practice a ratio of 75:25, though the exact ratio will depend on the viscosity of the fluid and on various design-related factors. Compared to the higher velocity jets characteristic of the catheters discussed in background, the low velocity of the fluid discharged from opening 331A and the cloud-like dispersion from porous section 200 greatly diminish the likelihood of dissection of tissue and dislodgement of plaque from the walls of vessels. When the injection is completed, the conically-shaped valve 310 will return to its original shape and the opening 331A to its original size.

The clinical benefit of such a dynamic restrictor/tip is threefold. First, the opening 331A of restrictor 300 can be made larger because its diameter is designed to decrease during an injection. This reduces drag on a guidewire during insertion, and allows for more accurate measurement of pressure in the vessel or other structure into which the tip of the catheter is inserted. One key design tradeoff is making the V-shape of restrictor 300 pliable enough to pass a guidewire but not pliable enough to evert under the hydraulic pressure created during injections. Second, the inward trumpet shape of conical wall portion 330 provides a centering mechanism for backloading a guidewire. Unlike the restrictor of FIG. 5A in which a 0.889 mm (0.035 inch) guidewire would be inserted into a 0.229 mm (0.009 inch) opening, restrictor 300 for a 4 French catheter 100 preferably has an opening of 0.965 mm (0.038 inch) at its distal end which preferably tapers to 0.229 mm (0.009 inches) at its apex 331. This should make the loading of the guidewire easier as restrictor 300 provides a conical taper in the direction of insertion. Lastly, if opening 331A should ever collapse entirely such that flow of fluid is blocked distally, then porous section 200 becomes the sole exit for the fluid, and the cloud-like dispersion that emanates radially from the microholes 220n will be maximized.

The stem of catheter 100 is preferably constructed of a semi-rigid plastic material that is softer than, and preferably thermally bonded to, the shaft. It is preferably made of a nylon material with a durometer of about 63 D, though it may range approximately from 45 D to 75 D. The stem can be shaped to the desired geometric configuration including, for example, Judkins Right (JR) and Judkins Left (JL) shapes for the coronary arteries; the Pigtail Straight and Angulated shapes for the ventricles and the aorta; the Visceral, the Cobra, and the RDC shapes for the renal arteries; and the Simmons, the JB, and the Headhunter configurations for catheterizations of the carotid arteries. If necessary, the section of the stem proximally adjacent to porous section 200 could be made of a stronger material relative to the strength of the material of the porous section.

In manufacturing the catheters of the present invention, the microholes may be incorporated or otherwise placed into the catheter as a secondary operation, preferably using a laser. Laser machining can make micron-sized holes that are very uniform and free of residual material. Additionally, laser machining can drill closely-spaced microholes very rapidly in any geometric pattern. FIGS. 4D and 4F, for example, each illustrate a repeating geometric pattern. A recurring pattern permits the use, e.g., of a single mask to drill many (e.g., a row of) microholes simultaneously. A pattern could then be achieved merely by alternately rotating the catheter to next position and then laser drilling the microholes, and continuing until the desired pattern is completed.

The restrictor 300 of catheter 100 is preferably made of a highly elastic plastic whose circular base portion 320 is bonded or otherwise affixed to the distal end of the stem. The circular base portion 320 acts as an extension of the stem but is made from a softer material. In its preferred manifestation, the tip material would be a 35 D nylon but could range approximately from 25 D to 55 D. The use of such lower durometer materials which are softer and more elastic enable the tip not only to be easily routed through the vasculature or other regions with far less risk of trauma to tissue but also to expand to accommodate passage of a guidewire in either direction as noted above.

FIGS. 5A-5B illustrate a catheter, generally designated 110, according to a second embodiment of the invention. This catheter includes a stem equipped with a porous section 200 along with a restrictor 400 affixed to the distal end of the stem.

Like the previous embodiment, the porous section 200 includes a large plurality of microholes 220n, each of which in communication with the lumen of the catheter. Although generally set between approximately 5 to 125 microns, the preferred diameter for the microholes 220n is about 50 microns, with all microholes 220n preferably having the same diameter. As best shown in FIG. 5B, the microholes are angled in the proximal direction. The degree of angularity can range approximately from 0 to 45 degrees, with a preferred angle of 20 degrees, though the exact angle will depend on the factors noted above. The preferred length of porous section 200 is 6 mm, though it can range from 2 mm to 2 cm or even longer. The microhole pattern is preferably located close to the tip/stem interface, as best shown in FIG. 5A, with a preferred spacing of less than 2 mm. The preferred microhole pattern is similar to that shown in FIG. 4E, with the number of microholes preferably being approximately n=640. In essentially most respects, the microhole pattern for catheter 110 can generally take the form of any of those disclosed in connection with the previous and subsequent embodiments.

The restrictor 400 takes the form of a hemispheric cap, which is preferably made of a highly elastic plastic. The cap features or otherwise defines an opening or endhole 431A, which should be smaller than the lumen of the stem. In a 4 French catheter 110, for example, the opening 431A would preferably have a diameter in the range from approximately 0.889 mm (0.035 inches) down to 0.0254 mm (0.001 inches), with a preferred dimension of 0.3302 mm (0.013 inches) as shown in FIG. 5B. In its preferred dimension, the opening 431A can expand to accommodate guidewires of up to 0.9652 mm (0.038 inches) due to the elasticity of restrictor 400. Upon removal of the guidewire, the opening 431A would return to its original diameter and then act as a fluid restrictor during injections of fluid. The presence of opening 431A also allows the pressure to be measured in the vessel into which tip 400 is inserted, and it further allows the doctor or other medical practitioner to determine whether the tip 400 is embedded in the wall of the vessel. This is important as it enables the practitioner to substantially reduce the likelihood of dissection or perforation of tissue. The restrictor 400 of catheter 110 is preferably 3 mm in length, although it may range from approximately 1 to 10 mm or other length depending upon the use to which catheter 110 is to be put.

Once catheter 110 is guided though the anatomy and its distal segment is properly positioned at the desired location, the injector or other pump to which the hub is connected will be activated to pressurize the fluid to be administered. This causes the fluid to flow through the lumen of catheter 110 and ultimately to the tip 400. More specifically, the pump causes the fluid to flow into the hub, through the shaft and stem, and into the distal segment of the catheter 110. Once the fluid reaches the distal segment, fluid begins to flow out of opening 431A and pressure begins to build against the proximal side of the hemispheric cap. Due to the size of its opening 431A, however, the restrictor 400 acts as a flow diverter. More specifically, as pressure increases, the amount of fluid flowing out of opening 431A increases initially, with little or no fluid exiting microholes 220n. As pressure increases further, however, progressively more fluid exits the microholes 220n and less exits the opening 431A because the structure of restrictor 400 limits the extent to which opening 431A can expand. From the opening 431A of restrictor 400 and, to a greater extent, the microholes 220n, the fluid then flows as a very fine, cloud-like dispersion into the targeted vessel, chamber or cavity.

Despite the relatively high pressure within its lumen, catheter 110 ensures the stability of its distal end and discharges therefrom a very fine dispersion of the fluid at very low velocities. Similar to the previous embodiment, the stability of catheter 110 during an injection is achieved by balancing the fluidic forces both axially and radially. Recoil or axial movement of catheter 110 is avoided because the force of the fluid flowing out of opening 431A in the distal direction is effectively counterbalanced by the cumulative force of the fluid flowing out of the inclined microholes 220n in the proximal direction. Whipping of catheter 110 is avoided because the forces of the fluid flowing radially out of the porous section 200 are substantially balanced due to the uniform distribution of the microholes 220n about its circumference. Consequently, the distal end of catheter 110 will remain exceptionally still in the vessel, chamber or cavity in which it is placed.

In the preferred implementation of catheter 110, the ratio of the fluid flowing out of opening 431A to that out of microholes 220n can be made quite close to that for catheter 100, for example, 25% and 75%, respectively. The exact ratio will depend on the viscosity of the fluid and on the design-related factors noted above. Compared to the higher velocity jets characteristic of prior art catheters, the low velocity of the fluid discharged from opening 431A and the cloud-like dispersion from porous section 200 greatly diminish the likelihood of dissection of tissue and dislodgement of plaque. The construction of catheter 110 and the composition of the various parts can be carried out in much the same way as described in connection with catheter 100.

As a related alternative, the restrictor of the present invention may be configured without an opening, thus completely preventing the flow of fluid from the distal end of the catheter. In this alternative, the microholes 220n would be oriented perpendicular to the stem, which would provide balance to the radial forces of injection. Made according to this alternative, a catheter would thus avoid whipping as well as recoil. Such an alternative would not only simplify the design but also reduce manufacturing costs.

FIGS. 6A-6C illustrate a catheter, generally designated 120, according to a third embodiment of the invention. This catheter includes a stem along with a restrictor 500 affixed to the distal end of the stem. Catheter 120 is similar to the other disclosed embodiments in that it uses a combination of proximally-angled microholes and a restrictor to create a uniform, fog-like dispersion of fluid during an injection while the tip remains stationary in the vessel or other structure into which it has been placed.

Like the previous embodiments, the stem has a porous section 200 that features a large plurality of microholes 220n, each of which in communication with the lumen of the catheter. Unlike those embodiments, however, the microholes of catheter 120 are situated not only in the stem but also in the restrictor 500. The microholes of restrictor 500 are generally designated in the drawings as 520n.

The microholes 220n of the stem have a diameter generally set between approximately 5 to at least 125 microns, with the preferred diameter being about 50 microns. All microholes preferably have the same diameter, and are angled in the proximal direction as best shown in FIG. 6C. For the illustrated embodiment, the angle can range approximately from 0 to 45 degrees, with a preferred angle of 20 degrees. It should be understood, however, that the exact angle(s) for any given configuration (of microholes for stem and/or restrictor) is that which substantially balances the forces of fluid flow in the axial and radial directions to avoid recoil and whipping.

The restrictor 500 in this embodiment takes the form of a spherical cap 501A having at its proximal end a cylindrical structure 501B that is bonded or otherwise affixed to the distal end of the stem. Preferably made of a highly elastic plastic, the spherical cap defines a cavity 531 and a distal opening or endhole 531A, the latter being preferably smaller than the lumen of the stem. In a 4 French catheter 120, for example, the opening 531A would preferably have a diameter in the range from approximately 0.889 mm (0.035 inches) down to 0.0254 mm (0.001 inches), with a preferred dimension of 0.3302 mm (0.013 inches) as shown in FIG. 6C. In its preferred dimension, the opening 531A can expand to accommodate guidewires of up to 0.9652 mm (0.038 inches) due to the elasticity of spherical cap 501A. Upon removal of the guidewire, the opening 531A would return to its original diameter and then act as a fluid restrictor during injections of fluid. The presence of opening 531A also allows the pressure to be measured in the vessel into which the tip 500 is inserted, and it further allows the doctor or other medical practitioner to determine whether the tip 500 is embedded in or flush against the wall of the vessel. This is important as it enables the practitioner to substantially reduce the likelihood of dissection or perforation of tissue.

The outside diameter of the spherical cap may be up to 50% greater than the outer diameter of the stem, though it is preferably 10% greater. For the 4 French catheter 120 shown in FIG. 6C, for example, this works out approximately to a 1.372 mm (0.054 inch) outer diameter for the stem and a 1.524 mm (0.060 inch) diameter for spherical cap 501A, a difference of 10% or 0.1524 mm (0.006 inches). The length of the restrictor 500 preferably ranges approximately from 3 to 10 mm, with a preferred length of 5 mm.

The microholes 520n in the spherical cap 501A and the microholes 220n in the stem are preferably deployed according to the one or more of the patterns specifically disclosed herein. Alternatively, the microholes may be deployed according to other patterns, with the ultimate goal being that the forces of fluid flow are substantially balanced in both the axial and radial directions to avoid recoil and whipping of the catheter 120.

FIGS. 6A-6D show a microhole pattern for catheter 120 in which the microholes more or less uniformly distributed about the distal end of the stem and the proximal side of spherical cap 501A. FIG. 6E shows an alternative pattern for the microholes, one which is divided into three substantially equal sections. Similar to the pattern shown in FIG. 4F, the most proximal section would contain the fewest number of microholes. The second section would contain twice as many microholes as the first section, and the third section would contain three times as many microholes as the first section. Unlike the first and second sections, however, the third section preferably would be not in the stem of catheter 120 but preferably on the proximal side of spherical cap 501A. As for how this particular microhole pattern would affect the function of the present embodiment, the increase in hole density toward the distal end would tend to decrease the hydraulic resistance of the sideholes, and that would offset the decreased pressure of the fluid as it flows axially through the catheter. This permits a more uniform distribution of flow through the microholes. The preferred pattern for this embodiment has n=648 sideholes, as shown in FIG. 6E. Depending on design constraints, however, it may be necessary to put fewer holes in the stem. At a minimum, it is preferred that at least 10% of the angled holes be deployed on the proximal side of spherical cap 501A.

The clinical benefit of a bulbous tip with sideholes is threefold. First, a spherically shaped restrictor 500 is less likely to become embedded in the wall of a vessel. This is because a spherical shape will always impinge on a flat surface at an oblique angle. Second, the increased cross-sectional area of the bulbous tip slows the flow of fluid, which increases static pressure and distributes the fluidic forces more uniformly across the microholes. Lastly, by increasing the angle of microholes 520n in spherical cap 501A even more, a greater reward angle is realized, thus providing a greater counterbalancing hydraulic force with which to resist the rearward forces created by the fluid flowing out of distal opening 531A. This enables the microholes 220n in the stem to be inclined at a significantly smaller angle, perhaps even as low as 0 degrees. The advantage of such an orientation is that the fluid exiting the microholes would have less blow back, i.e., less motion directed away from the region to be imaged. For example, if the left coronary artery were engaged, there would be less fluid blow back into the aorta.

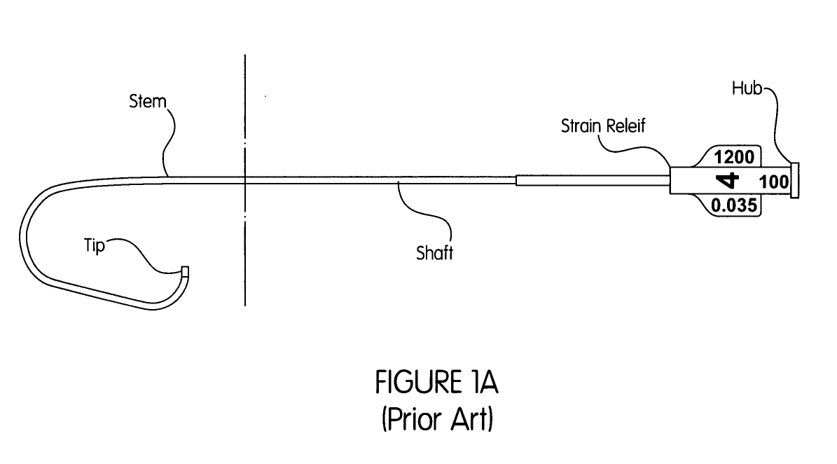

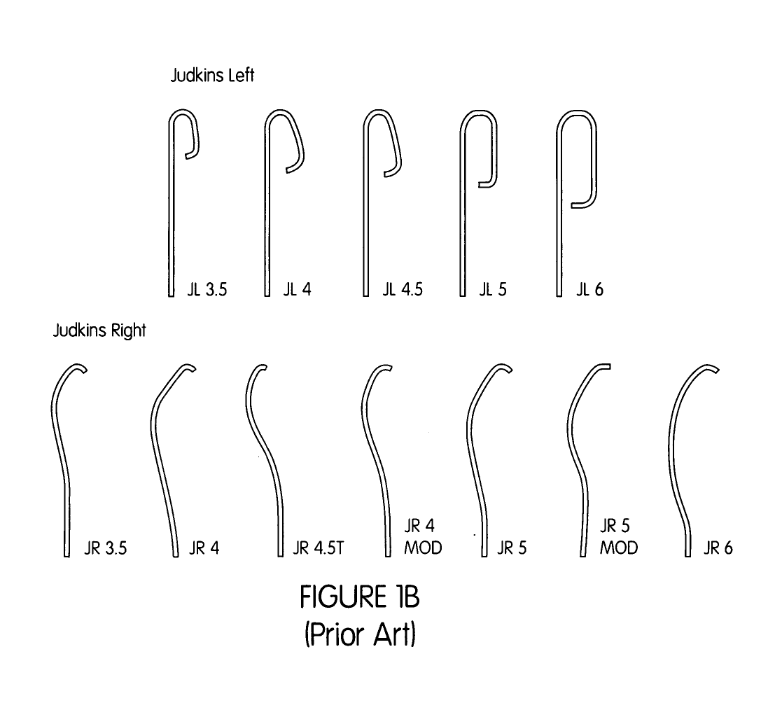

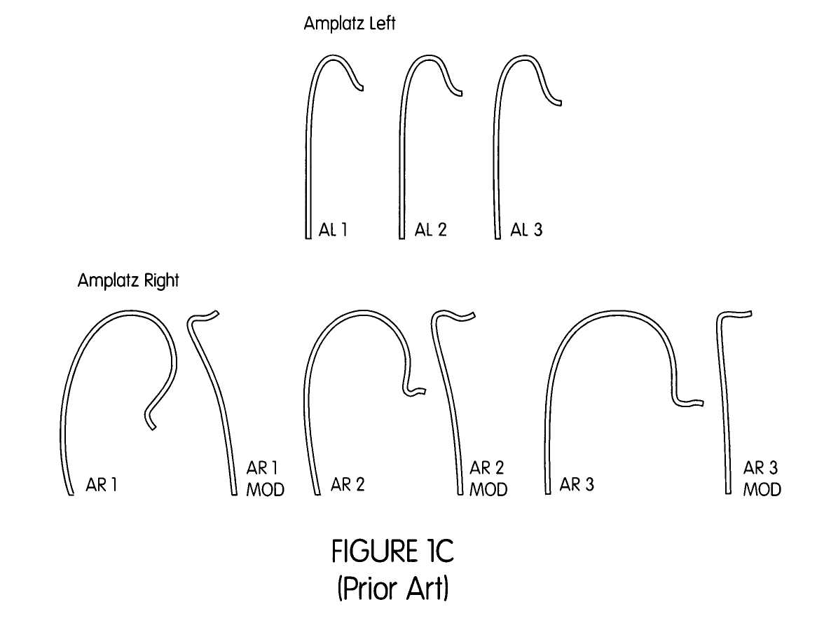

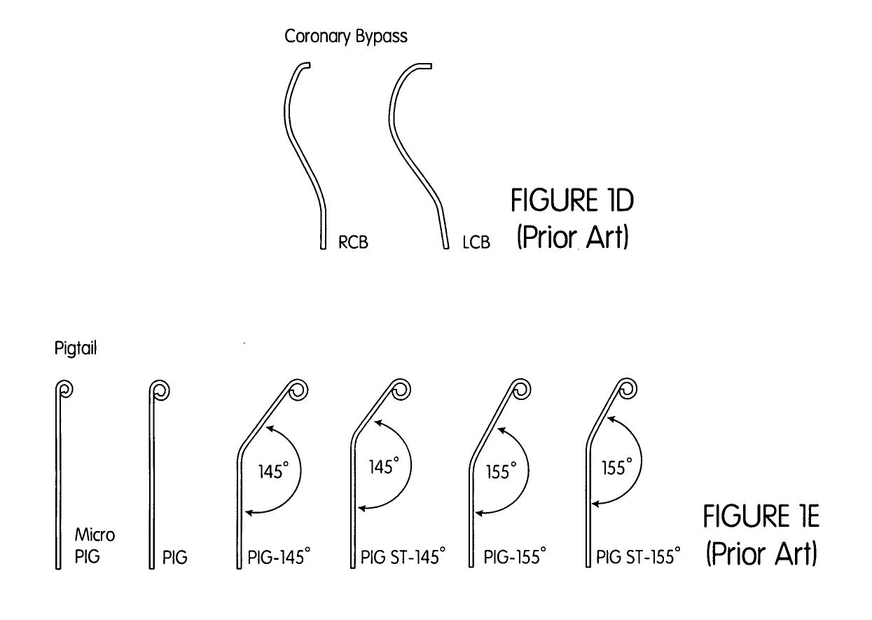

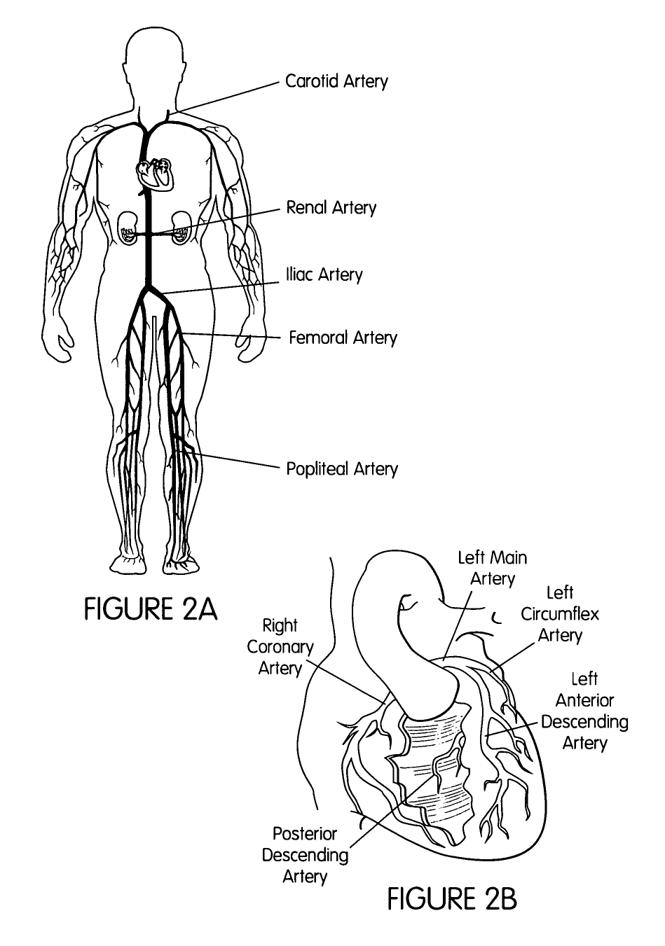

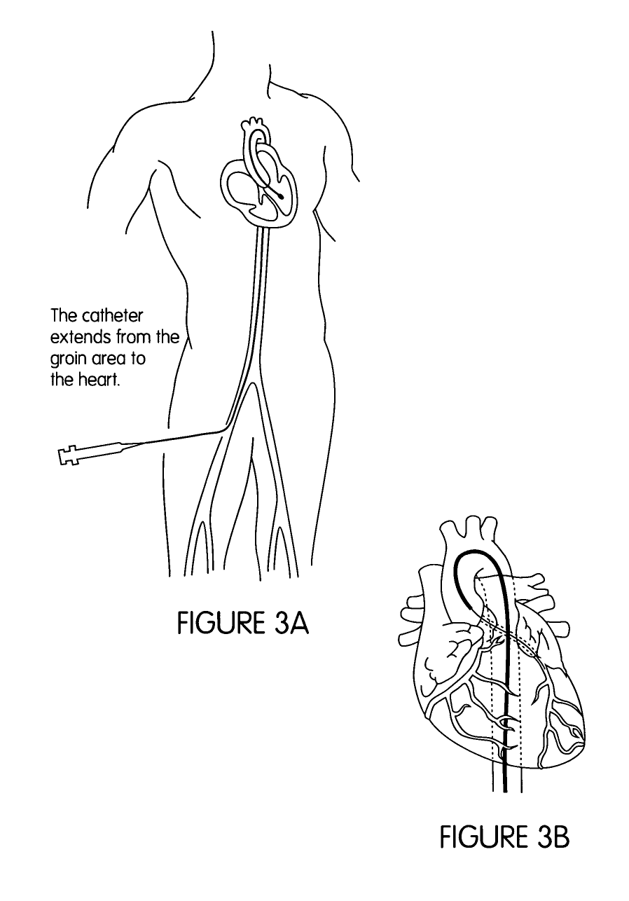

FIGS. 7A-7H illustrate a catheter, generally designated 130, according to a fourth embodiment of the invention. This embodiment is primarily directed to a flush-type catheter, which is a conventional design generally capable of quickly delivering a large volume of fluid and is thus ideal for delivering contrast fluid to a large chamber such as a ventricle as a prerequisite to imaging same (i.e., a ventriculogram). A pigtail catheter is one example of a flush-type catheter, examples of which are shown in FIG. 1E, so this embodiment is described herein primarily in that context. The reader should understand, however, that the features disclosed below may also be applied or adapted to catheters of other types, shapes, sizes, and purposes.

The catheter 130 includes a restrictor essentially identical to that disclosed in connection with the 4 and 5 French catheters 100 shown in FIGS. 4K-4N and 4O-4R, respectively. Catheter 130, however, has a stem whose porous section 250 is different in several respects as compared to the other disclosed embodiments. These differences are largely due to the function of a flush-type catheter for which porous section 250 is intended. In the catheter of this embodiment, these differences are manifested mostly in the form of the shape and length of the porous section, the different type of microhole pattern applied to the porous section, and the larger diameter and angle of the microholes in that pattern.

FIGS. 7A and 7E show the distal segment of the pigtail-shaped catheter 130 in a 4 and a 5 French size, respectively. In these views, the distal segment of catheter 130 has been uncoiled (i.e., straightened from its normal pigtail shape). For both the 4 and 5 French versions, the stem is approximately 59.055 mm in length. As shown in FIG. 7B, the approximate outer and inner (lumen) diameters of the 4 French catheter 130 are 1.372 mm and 0.965 mm, respectively. The approximate outer and inner diameters of the 5 French catheter 130 are 1.702 mm and 1.219 mm, respectively, as shown in FIG. 7F.

Preferably located in proximity to the distal end of the stem, the porous section 250 includes a large plurality of microholes 250n, each of which in communication with the lumen of the catheter 130. All microholes 250n preferably have the same diameter. Although the diameter is generally best set between approximately 5 to 250 microns, the preferred diameter for the microholes 250n is about 100 microns, which is about twice the size recommended for the first embodiment. This larger diameter is shown in FIGS. 7D and 7H as being 0.101 mm and 0.102 mm for catheters 130 of 4 and 5 French size, respectively. Unlike catheter 100, the microholes are preferably not inclined in this embodiment, i.e., they make a zero degree angle in the proximal direction with respect to a plane normal to the longitudinal axis of catheter 130.

FIGS. 7C and 7G show the preferred microhole pattern in greater detail for catheter 130 in a 4 and a 5 French size, respectively, with the number of microholes 250n preferably being approximately n=360. In these views, the porous section 250 of catheter 130 has been not only been uncoiled but also unwrapped to a flat sheet from its normal cylindrical shape. The length of porous section 250 is approximately 50 mm, and its pattern is spaced approximately 2.032±0.508 mm from restrictor 300. FIG. 7D shows the circumference for the 4 French catheter to be approximately 4.3078 mm, and FIG. 7H shows a circumference of 5.347 mm for the 5 French catheter 130.

FIGS. 7C and 7G illustrate that the preferred microhole pattern for catheter 130 takes the form of two laterally-spaced spiral formations 230A and 230B, with the spacing therebetween being approximately 2.1535 mm and 2.673 mm for the 4 and 5 French versions, respectively. Each spiral formation features a plurality of laterally-offset rows of microholes 250n, with each row preferably comprising about 10 microholes. The rows in the 4 French catheter 130 are offset by approximately 0.3589 mm as suggested in FIG. 7D, and those in the 5 French catheter 130 are offset by approximately 0.445 mm as suggested in FIG. 7H. Furthermore, in each spiral formation, each row is separated longitudinally from its neighbor by approximately 0.2540 mm as shown in FIGS. 7D and 7H. Ultimately, the two spiral formations are laid out so that each row in one spiral formation 230A/230B is deployed 180 degrees about the cylindrical stem from its counterpart row in the other spiral formation 230B/230A. In addition, the microholes in each row are spaced apart in the longitudinal direction by approximately 0.254 mm. This pattern is well suited for a pigtail configuration because it permits a large bolus of fluid to be delivered rapidly to a large volume such as a ventricle of the human heart.

Once catheter 130 is guided though the anatomy and its distal segment properly positioned within a ventricle or other structure, the injector or other pump to which the hub is connected will be activated to pressurize the fluid to be administered. This causes the fluid to flow through the lumen of catheter 130 and ultimately to the tip. Once the fluid reaches the distal segment, fluid begins to flow out of opening 331A and pressure begins to build against the proximal side of conically shaped valve 310. Increased hydraulic pressure, however, will be required to push the fluid through the microholes 250n of porous section 250. As soon as the pressure reaches the design-dependent threshold, the conical wall portion 330 begins to flatten out distally thereby decreasing the size of opening 331A. In response to the decreasing size of opening 331A, the amount of the fluid flowing out of opening 331A decreases while the fluid flowing out microholes 250n of porous section 250 increases accordingly. From the opening 331A of restrictor 300 and, to a greater extent, the microholes 250n, the fluid then flows as a very fine, cloud-like dispersion into the ventricle or other targeted structure.

Despite the relatively high pressure within its lumen, catheter 130 ensures the stability of its distal end and discharges therefrom a very fine dispersion of the fluid at very low velocities. The stability of catheter 130 during an injection is achieved by balancing the fluidic forces both radially and axially. Whipping of catheter 130 is avoided because the forces of the fluid flowing radially out of the microholes 250n are substantially balanced due to the deployment of the spiral formations 230A and 230B about the stem, particularly because each row in one spiral formation 230A/230B is diametrically opposite from its counterpart row in the other spiral formation 230B/230A. Recoil is effectively addressed because the force of the fluid flowing axially is largely spent in trying to uncoil the pigtail and to flatten out conically-shaped valve 310, with the fluid that emerges from opening 331A exiting at relatively low velocity. The distal end of catheter 130 will thus be relatively motionless in the ventricle or other structure in which it is placed.

Catheter 130 can be configured so that 90% or more of the fluid is very finely dispensed through the microholes 250n in a cloud-like form, with the remainder exiting the distal opening 331A at a very low velocity. Alternatively, the percentage of the fluid flowing out of the microholes versus that out of the opening could be set at 60% to 40%, respectively, or even lower. Used with standard contrast media, catheter 130 has exhibited in practice a ratio of 80:20, though the exact ratio will depend on the viscosity of the fluid and the design-related factors noted above. Compared to the higher velocity jets characteristic of prior art catheters, the low velocity of the fluid discharged from opening 331A and the cloud-like dispersion from porous section 250 greatly diminish the likelihood of tissue irritation or damage. Minimizing such trauma is particularly important during a ventriculogram to prevent electrophysiological abnormalities such as premature ventricular contractions (PVCs). The construction of catheter 130 and the composition of the various parts can be carried out in much the same way as described in connection with catheter 100.

As a related alternative, the restrictor of this embodiment may be configured without an opening, thus completely preventing the flow of fluid from the distal end of the catheter. In this alternative, the microholes 250n would be oriented perpendicular to the stem, which would provide balance to the radial forces of injection. Made according to this alternative, a catheter would thus avoid whipping and recoil. Such an alternative would not only simplify the design but also reduce manufacturing costs.

The catheters of the present invention have a large number of microholes, preferably near the distal end. The purpose of the microholes is to create a dispersion of fine droplets of contrast fluid that envelop the distal end of the catheter to maintain a more stable tip position during injections and provide better image quality. The fog of contrast fluid produced by these catheters has three clinically beneficial effects. First, it reduces the kinetic energy of the fluid thereby decreasing the likelihood of tissue trauma. Second, it enhances image quality by creating a more uniform bolus of fluid around the catheter rather than a jet discharged from the tip. Particularly with regard to catheter 100, this permits imaging of the ostial region of a vessel, which is not possible with prior art angiographic catheters and which will possibly reduce the amount of contrast fluid required during such a procedure. Third, it increases stability of the tip by distributing the hydraulic forces more uniformly over the distal end of the catheter.

Several embodiments and related aspects for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations that fall within the literal meaning, and range of equivalency, of the claims are to be embraced within their scope. Persons of such skill will also recognize that the scope of the invention is indicated by the claims rather than by any particular example or embodiment discussed in the foregoing description.

Accordingly, to promote the progress of science and useful arts, we secure by Letters Patent exclusive rights to all subject matter embraced by the following claims for the time prescribed by the Patent Act.

What is claimed is:

1. A catheter assembly for introducing fluid into a vessel, the catheter assembly comprising:
(a) a shaft;
(b) a hub affixed to a proximal end of said shaft;
(c) a stem affixed to a distal end of said shaft, said stem having a porous section approximate a distal end thereof, said porous section defining a plurality of microholes generally distributed uniformly thereabout and inclined by a predetermined angle in a proximal direction; and
(d) a tip affixed to said distal end of said stem, said tip including a conically-shaped valve with an apex thereof defining an opening and pointing in the proximal direction;
such that, as the fluid flows within said catheter assembly and pressure increases within said tip, said conically-shaped valve dynamically changing and thus generally decreasing a size of said opening so that the amount of the fluid flowing out of (A) said opening of said tip decreases and (B) said microholes of said stem increases, with the forces of the fluid flowing out of said microholes and said opening substantially balancing thereby enabling a position of said tip and said stem within the vessel to remain stable while fluid is finely dispersed therefrom.

2. The catheter assembly of claim 1 wherein an outer part of said tip is made of a nylon in a range approximately from 25 D nylon to 55 D nylon.

3. The catheter assembly of claim 2 wherein said outer part of said tip is made of 35 D nylon.

4. The catheter assembly of claim 1 wherein a length of said tip ranges approximately from 1 mm to 10 mm.

5. The catheter assembly of claim 4 wherein said length of said tip ranges from approximately 1 mm to 2 mm.

6. The catheter assembly of claim 1 wherein a size of said opening of said conically-shaped valve ranges approximately from 0.889 mm at a base thereof to 0.1016 mm at said apex in absence of fluid pressure.

7. The catheter assembly of claim 6 wherein said size of said opening at said apex is approximately in the range of 0.220 mm to 0.260 mm.

8. The catheter assembly of claim 1 wherein said conically-shaped valve includes:
(a) a circular base portion affixed to approximately a distal end of said tip; and
(b) a conical wall portion extending and decreasing in thickness from said circular base portion to said apex.

9. The catheter assembly of claim 8 wherein a size of said opening of said tip therein ranges from approximately 0.889 mm at said circular base portion to approximately 0.1016 mm at said apex in absence of fluid pressure.

10. The catheter assembly of claim 6 wherein said size of said opening at said apex is approximately in the range of 0.220 mm to 0.260 mm.

11. The catheter assembly of claim 1 wherein a difference in a size of said opening of said conically-shaped valve between an absence of pressure and a maximum pressure within said tip ranges approximately from 0.0762 mm to 0.127 mm.

12. The catheter assembly of claim 1 wherein a difference in a size of said opening of said conically-shaped valve between an absence of pressure and a maximum pressure depends on at least one of a shape of said valve and a thickness of a wall portion of said valve.

13. The catheter assembly of claim 1 wherein said conically-shaped valve is made of a material(s) sufficiently pliable to enable passage of a guidewire therethrough but to avoid everting under the pressure extant within said tip.

14. The catheter assembly of claim 1 wherein said stem is made of a nylon in a range approximately from 45 D nylon to 75 D nylon.

15. The catheter assembly of claim 14 wherein said stem is made of 63 D nylon.

16. The catheter assembly of claim 1 wherein said predetermined angle depends on at least one of a size of said catheter assembly, a shape of said catheter assembly, a desired volume of the fluid to be introduced into the vessel, and a ratio of an amount of the fluid to be flowing out of said microholes to that to be flowing out of said opening.

17. The catheter assembly of claim 1 wherein said predetermined angle by which said microholes of said porous section are inclined ranges approximately from 0 to 45 degrees.

18. The catheter assembly of claim 17 wherein said predetermined angle by which said microholes of said porous section is inclined is approximately 20 degrees.

19. The catheter assembly of claim 17 wherein said predetermined angle by which said microholes of said porous section is inclined changes with position along said stem.

20. The catheter assembly of claim 1 wherein a size of said microholes is in a range approximately from 5 microns to 250 microns.

21. The catheter assembly of claim 20 wherein said size of said microholes is approximately 50 microns.

22. The catheter assembly of claim 1 wherein said microholes are distributed about said porous section according to a pattern having a plurality of pairs of longitudinally arranged rows, with each of said row pairs being laterally spaced generally equidistantly from its neighbors.

23. The catheter assembly of claim 1 wherein a diameter of said microholes of said porous section changes with position along said stem.

24. The catheter assembly of claim 1 wherein said catheter assembly is for use with a guidewire.

25. The catheter assembly of claim 1 wherein said catheter assembly permits measurement of pressure extant in the vessel.

26. The catheter assembly of claim 1 further comprising a strain relief element interconnected between said hub and said proximal end of said shaft.

27. The catheter assembly of claim 1 wherein a ratio of the fluid flowing out of said opening to that out of said microholes is approximately 25% and 75%, respectively, when the pressure of the fluid has flattened out said conically-shaped valve.

28. The catheter assembly of claim 1 wherein a ratio of the fluid flowing out of said opening to that out of said microholes is between approximately 10% and 90%, respectively, and 49% and 51%, respectively, when the pressure of the fluid has flattened out said conically-shaped valve.

29. A catheter assembly for introducing fluid into a vessel, the catheter assembly comprising:
(a) a stem having approximate a distal end thereof a porous section defining a plurality of microholes distributed thereabout and inclined by a predetermined angle in a proximal direction; and
(b) a tip affixed to said distal end of said stem, said tip including a conically-shaped valve with an apex thereof pointing in the proximal direction and defining an opening whose size generally decreases as said conically-shaped valve dynamically changes as pressure of the fluid within said tip increases;
wherein the forces of the fluid flowing from within said catheter assembly out of said opening of said tip and out of said microholes of said stem substantially balance thereby substantially eliminating both recoil and whipping of said catheter assembly thus enabling a position thereof within the vessel to remain stable while the fluid is finely dispersed therefrom.

30. The catheter assembly of claim 29 wherein said stem is made of a nylon in a range approximately from 45 D nylon to 75 D nylon.

31. The catheter assembly of claim 30 wherein said stem is made of 63 D nylon.

32. The catheter assembly of claim 29 wherein said microholes are uniformly distributed about said porous section according to a pattern having a plurality of pairs of longitudinally arranged rows, with each of said row pairs being laterally spaced generally equidistantly from its neighbors.

33. The catheter assembly of claim 29 wherein said microholes are radially distributed about said porous section uniformly and according to a gradient along a longitudinal axis thereof.

34. The catheter assembly of claim 33 wherein said microholes along the longitudinal axis are deployed in a plurality of sections of substantially equal length wherein the number of said microholes in each of said sections changes according to a linear progression.

35. The catheter assembly of claim 34 wherein said plurality of sections includes a proximal section having a fewest number of microholes, a middle section having double the number of microholes in said proximal section, and a distal section having triple the number of microholes in said proximal section.

36. The catheter assembly of claim 29 wherein said microholes are distributed about said porous section according to a pattern having a plurality of laterally-spaced spiral formations.

37. The catheter assembly of claim 29 wherein said predetermined angle depends on at least one of a size of said catheter assembly, a shape of said catheter assembly, a desired volume of the fluid to be introduced into the vessel, and a ratio of an amount of the fluid to be flowing out of said microholes to that to be flowing out of said opening.

38. The catheter assembly of claim 29 wherein said predetermined angle by which said microholes of said porous section are inclined ranges approximately from 0 to 45 degrees.

39. The catheter assembly of claim 38 wherein said predetermined angle by which said microholes of said porous section is inclined is approximately 20 degrees.

40. The catheter assembly of claim 38 wherein said predetermined angle by which said microholes of said porous section is inclined is approximately 0 degrees.

41. The catheter assembly of claim 38 wherein said predetermined angle by which said microholes of said porous section is inclined changes with position along said stem.

42. The catheter assembly of claim 29 wherein a size of said microholes is in a range approximately from 5 microns to 250 microns.

43. The catheter assembly of claim 42 wherein said size of said microholes is approximately 50 microns.

44. The catheter assembly of claim 42 wherein said size of said microholes is approximately 100 microns.

45. The catheter assembly of claim 29 wherein a diameter of said microholes of said porous section changes with position along said stem.

46. The catheter assembly of claim 29 wherein an outer part of said tip is made of a nylon in a range approximately from 25 D nylon to 55 D nylon.

47. The catheter assembly of claim 46 wherein said outer part of said tip is made of 35 D nylon.

48. The catheter assembly of claim 29 wherein a length of said tip ranges approximately from 1 mm to 10 mm.

49. The catheter assembly of claim 48 wherein said length of said tip ranges approximately 1 mm to 2 mm.

50. The catheter assembly of claim 29 wherein a size of said opening of said conically-shaped valve ranges approximately from 0.889 mm at a base thereof to 0.1016 mm at said apex in absence of fluid pressure.

51. The catheter assembly of claim 50 wherein said size of said opening at said apex is approximately in the range of 0.220 mm to 0.260 mm.

52. The catheter assembly of claim 29 wherein said conically-shaped valve includes:
(a) a circular base portion affixed to approximately a distal end of said tip; and
(b) a conical wall portion extending and decreasing in thickness from said circular base portion to said apex.

53. The catheter assembly of claim 52 wherein a size of said opening of said tip therein ranges from approximately 0.889 mm at said circular base portion to approximately 0.1016 mm at said apex in absence of fluid pressure.

54. The catheter assembly of claim 53 wherein said size of said opening at said apex is approximately in the range of 0.220 mm to 0.260 mm.

55. The catheter assembly of claim 29 wherein a difference in a size of said opening of said conically-shaped valve between an absence of pressure and a maximum pressure within said tip ranges approximately from 0.0762 mm to 0.127 mm.

56. The catheter assembly of claim 29 wherein a difference in a size of said opening of said conically-shaped valve between an absence of pressure and a maximum pressure depends on at least one of a shape of said valve and a thickness of a wall portion of said valve.

57. The catheter assembly of claim 29 wherein said conically-shaped valve is made of a material(s) sufficiently pliable to enable passage of a guidewire therethrough but to avoid everting under the pressure extant within said tip.

58. The catheter assembly of claim 29 wherein said catheter assembly is for use with a guidewire.

59. The catheter assembly of claim 29 wherein said catheter assembly permits measurement of pressure extant in the vessel.

60. The catheter assembly of claim 29 further comprising:
(a) a shaft affixed to a proximal end of said stem;
(b) a strain relief element affixed to a proximal end of said shaft; and
(c) a hub affixed to a proximal end of said strain relief element.

61. The catheter assembly of claim 29 wherein a ratio of the fluid flowing out of said opening to that out of said microholes is approximately 25% and 75%, respectively, when the pressure of the fluid has flattened out said conically-shaped valve.

62. The catheter assembly of claim 29 wherein a ratio of the fluid flowing out of said opening to that out of said microholes is between approximately 10% and 90%, respectively, and 49% and 51%, respectively, when the pressure of the fluid has dynamically changed said conically-shaped valve.

63. A catheter assembly for introducing fluid into a vessel, the catheter assembly comprising a restrictor at a distal end thereof, said restrictor including a conically-shaped valve comprising:
(a) a circular base portion formed approximate a distal end of said restrictor; and
(b) a conical wall portion extending in a proximal direction from said circular base portion to an apex thereof, said apex defining an opening whose size generally decreases as said conically-shaped valve flattens out distally as pressure of the fluid within said restrictor increases.

64. The catheter assembly of claim 63 wherein said conical wall portion decreases in thickness in the proximal direction from said circular base portion to said apex.

65. The catheter assembly of claim 63 wherein a size of said opening ranges from approximately 0.889 mm at said circular base portion to approximately 0.1016 mm at said apex in absence of fluid pressure.

66. The catheter assembly of claim 65 wherein said size of said opening at said apex is approximately in the range of 0.220 mm to 0.260 mm.

67. The catheter assembly of claim 63 wherein a difference in a size of said opening of said conically-shaped valve between an absence of pressure and a maximum pressure within said restrictor ranges approximately from 0.0762 mm to 0.127 mm.

68. The catheter assembly of claim 63 wherein a difference in a size of said opening of said conically-shaped valve between an absence of pressure and a maximum pressure depends on at least one of a shape of said valve and a thickness of said conical wall portion.

69. The catheter assembly of claim 63 wherein said conically-shaped valve is made of a material(s) sufficiently pliable to enable passage of a guidewire therethrough but to avoid everting under the pressure extant within said restrictor.

70. A catheter assembly for introducing fluid into a vessel, said catheter assembly comprising:
(a) a stem having approximate a distal end thereof a porous section defining a plurality of microholes distributed thereabout and inclined by a predetermined angle in a proximal direction; and
(a) a restrictor affixed to said distal end of said stem, said restrictor defining an opening therein whose size generally decreases as pressure of the fluid within said restrictor increases;
wherein the forces of the fluid flowing from within said catheter assembly out of said opening of said restrictor and out of said microholes of said stem substantially balance to prevent axial and radial movement of said catheter assembly thus enabling a position thereof within the vessel to remain stable while the fluid is finely dispersed therefrom in a cloud-like form.

71. The catheter assembly of claim 70 wherein said microholes are generally distributed uniformly about said porous section both longitudinally along an axis thereof and radially about a circumference thereof.

72. The catheter assembly of claim 71 wherein said microholes are distributed according to a pattern having a plurality of pairs of longitudinally arranged rows, with each of said row pairs being laterally spaced generally equidistantly from its neighbors.

73. The catheter assembly of claim 71 wherein said predetermined angle by which said microholes of said porous section is inclined is approximately 20 degrees.

74. The catheter assembly of claim 70 wherein said microholes are distributed about said porous section according to a pattern having a plurality of laterally-spaced spiral formations.

75. The catheter assembly of claim 74 wherein said porous section has two of said spiral formations each of which having a plurality of laterally-offset rows of microholes, with each of said rows in one of said spiral formations being diametrically opposite from a counterpart one of said rows in the other of said spiral formations.

76. The catheter assembly of claim 74 wherein said predetermined angle by which said microholes of said porous section is inclined is approximately 0 degrees.

77. The catheter assembly of claim 74 wherein said catheter assembly is implemented as one of a pigtail catheter and an other flush-type catheter.

78. A catheter comprising a distal segment having:
(a) a porous section; and
(b) a restrictor contiguous with said porous section, said restrictor defining an opening therein whose size generally decreases as pressure of fluid within said restrictor increases.

79. The catheter of claim 78 wherein said porous section defines a plurality of microholes distributed thereabout.

80. The catheter of claim 79 wherein a diameter of said microholes of said porous section changes with position along said stem.

81. The catheter of claim 79 wherein said microholes are inclined by a predetermined angle in a proximal direction.

82. The catheter of claim 81 wherein said predetermined angle by which said microholes of said porous section is inclined changes with position along said stem.

83. A catheter comprising a restrictor approximate a distal end thereof, said restrictor defining an opening therein whose size generally decreases as pressure of fluid within said restrictor increases.

84. A catheter comprising:
(a) a shaft; and
(b) a stem affixed to a distal end of said shaft, said stem having a porous section defining a plurality of microholes.

85. The catheter of claim 84 wherein a size of said microholes is in a range approximately from 5 microns to 250 microns.

86. The catheter of claim 84 wherein said size of said microholes is approximately 50 microns.

87. The catheter of claim 84 wherein a diameter of said microholes of said porous section changes with position along said stem.

88. The catheter of claim 84 wherein a size of said microholes is in a range approximately from 5 microns to 125 microns.

89. The catheter of claim 84 wherein said microholes are uniformly distributed about said porous section of said stem.

90. The catheter of claim 84 wherein said microholes are radially distributed about said porous section uniformly and according to a gradient along a longitudinal axis thereof.

91. The catheter of claim 84 wherein said microholes are radially distributed about said porous section uniformly and longitudinally distributed via a plurality of sections of substantially equal length wherein the number of said microholes in each of said sections changes according to a linear progression.

92. The catheter of claim 91 wherein said plurality of sections includes a proximal section having a fewest number of microholes, a middle section having double the number of microholes in said proximal section, and a distal section having triple the number of microholes in said proximal section.

93. The catheter of claim 84 wherein said microholes are distributed about said porous section according to a pattern having a plurality of laterally-spaced spiral formations.

94. The catheter of claim 93 wherein said porous section has two of said spiral formations each of which having a plurality of laterally-offset rows of microholes, with each of said rows in one of said spiral formations being diametrically opposite from a counterpart one of said rows in the other of said spiral formations.

95. The catheter of claim 84 wherein said microholes are deployed about said porous section such that the forces of fluid flowing from within said catheter out said microholes thereof in a finely dispersed, cloud-like form are substantially balanced thereby substantially eliminating movement of said catheter and thus enabling a position thereof to remain exceptionally stable.

96. The catheter of claim 95 further including a restrictor attached to a distal end of said stem, said restrictor acting as a plug thereat and thus preventing flow therefrom.

97. The catheter of claim 84 further including a restrictor attached to a dismal end of said stem, said restrictor acting as a plug thereat and thus preventing flow therefrom.

98. The catheter of claim 84 further including a restrictor at a distal end thereof contiguous with said porous section, said restrictor defining an opening therein whose size generally decreases as pressure of fluid within said restrictor increases.

99. The catheter of claim 98 wherein said microholes are generally distributed uniformly about said porous section and inclined by a predetermined angle in a proximal direction such that the forces of fluid flowing from within said catheter out of said opening of said restrictor and out of said microholes of said porous section substantially balance thereby substantially eliminating movement of said catheter thus enabling a position thereof to remain exceptionally stable while the fluid is finely dispersed therefrom.

100. The catheter of claim 99 wherein said predetermined angle depends on at least one of a size of said catheter, a shape of said catheter, a desired volume of the fluid to be injected, and a ratio of the fluid to be flowing out of said microholes to that to be flowing out of said opening.

101. The catheter of claim 99 wherein said predetermined angle by which said microholes of said porous section is inclined ranges approximately from 0 to 45 degrees.

102. The catheter of claim 101 wherein said predetermined angle by which said microholes of said porous section is inclined is approximately 20 degrees.

103. The catheter of claim 101 wherein said predetermined angle by which said microholes is inclined changes with position along said stem.

104. The catheter of claim 99 wherein a size of said microholes is in a range approximately from 5 microns to 250 microns.

105. The catheter of claim 104 wherein said size of said microholes is approximately 50 microns.

106. The catheter of claim 99 wherein a size of said microholes is in a range approximately from 5 microns to 100 microns.

107. The catheter of claim 84 further including a restrictor at a distal end thereof contiguous with said porous section, said restrictor being manifested as a hemispheric cap that defines an opening at a distal end thereof.

108. The catheter of claim 107 wherein said microholes are generally distributed uniformly about said porous section and inclined by a predetermined angle in a proximal direction such that the forces of fluid flowing from within said catheter out of said opening of said restrictor and out of said microholes of said porous section substantially balance thereby substantially eliminating movement of said catheter thus enabling a position thereof to remain exceptionally stable while the fluid is finely dispersed therefrom.

109. The catheter of claim 108 wherein said predetermined angle by which said microholes of said porous section is inclined ranges approximately from 0 to 45 degrees.

110. The catheter of claim 108 wherein a size of said microholes is in a range approximately from 5 microns to 125 microns.

111. The catheter of claim 110 wherein said size of said microholes is approximately 50 microns.

112. The catheter of claim 84 further including a restrictor at a distal end thereof contiguous with said porous section, said restrictor comprising a spherical cap defining a cavity therein and an opening at a distal end thereof and also a plurality of microholes on a proximal side thereof.

113. The catheter of claim 112 wherein said microholes of said porous section are inclined by a predetermined angle in a proximal direction such that the forces of fluid flowing from within said catheter out of said opening of said restrictor and out of said microholes of said porous section and said spherical cap substantially balance thereby substantially eliminating movement of said catheter thus enabling a position thereof to remain exceptionally stable while the fluid is finely dispersed therefrom.

114. The catheter of claim 113 wherein said predetermined angle by which said microholes of said porous section is inclined ranges approximately from 0 to 45 degrees.

115. The catheter of claim 113 wherein said predetermined angle by which said microholes of said porous section is inclined changes with position along said stem.

116. The catheter of claim 113 wherein a size of said microholes is in a range approximately from 5 microns to 125 microns.

117. The catheter of claim 116 wherein said size of said microholes is approximately 50 microns.

118. An injector system comprising:
(a) an injector for injecting a fluid into a patient; and
(b) a catheter operably associated with said injector for introducing the fluid into a bodily structure, said catheter comprising:
(I) a porous section; and
(II) a restrictor contiguous with said porous section, said restrictor defining an opening therein whose size generally decreases as pressure of fluid within said restrictor increases.

119. The injector system of claim 118 wherein said porous section defines a plurality of microholes distributed thereabout and inclined by a predetermined angle in a proximal direction.

120. The injector system of claim 118 wherein said restrictor comprises a conically-shaped valve with an apex thereof pointing in the proximal direction and defining an opening whose size generally decreases as pressure of the fluid within said tip increases.

121. The injector system of claim 118 wherein the forces of the fluid flowing out of said opening and said porous section substantially balance to allow said catheter to remain stable within the bodily structure.
